(19) United States
(12) Patent Application Publication
(10) Pub. No.: US 2008/0045489 A1
(43) Pub. Date: Feb. 21, 2008

(54) 3,4-DI-SUBSTITUTED CYCLOBUTENE-1,2-DIONES AS CXC-CHEMOKINE RECEPTOR LIGANDS

(76) Inventors: Jianhua Chao, Pompton Lakes, NJ (US); Michael F. Czarniecki, Watchung, NJ (US); Zhenmin He, Shanghai (CN); Gaifa Lai, Monmouth Junction, NJ (US); J. Robert Merritt, Ewing, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/773,479

(22) Filed: Jul. 5, 2007

Related U.S. Application Data
(60) Provisional application No. 60/819,541, filed on Jul. 7, 2006.

(51) Int. Cl. A61K 31/5355; A61K 31/192; A61K 31/34; A61K 31/341; A61K 31/5415; A61P 1/00; A61P 11/06; A61P 17/06; A61P 25/28; C07D 413/10; C07D 307/02; C07D 205/04; A61P 9/00; A61P 43/00; A61P 35/00; A61P 31/12; A61P 31/04; A61P 29/00; A61P 25/00; A61P 17/00; A61P 11/00; A61K 31/573; A61K 31/40; A61K 31/415; A61K 31/519

(52) U.S. Cl. 514/171; 514/210.18; 514/226.5; 514/231.5; 514/262.1; 514/406; 514/422; 514/471; 514/570; 544/152; 548/517; 548/953; 549/492

(57) ABSTRACT

Disclosed are novel cyclobutenedione Compounds 1 to 18 comprising a cyclobutenedione ring, a substituted phenyl ring, and a —CH(C₂H₅)-furan moiety. The phenyl ring and the —CH(C₂H₅)-furan moiety are each bound to the cyclobutenedione ring through a —NH— moiety. Also disclosed are methods of treating chemokine mediated diseases (e.g., cancer, COPD, acute and chronic inflammatory disorders, psoriasis, cystic fibrosis, and asthma) using the novel Compounds 1 to 18.

### REFERENCE TO RELATED APPLICATION

This application claims the benefit of U.S. Provisional Application Ser. No. 60/819,541 filed Jul. 7, 2006.

### FIELD OF THE INVENTION

The present invention relates to novel substituted cyclobutenedione compounds, pharmaceutical compositions containing the compounds, and the use of the compounds and formulations in treating CXC chemokine-mediated diseases.

### BACKGROUND OF THE INVENTION

Chemokines are chemotactic cytokines that are released by a wide variety of cells to attract macrophages, T-cells, eosinophils, basophils, neutrophils and endothelial cells to sites of inflammation and tumor growth. There are two main classes of chemokines, the CXC-chemokines and the CC-chemokines. The class depends on whether the first two cysteines are separated by a single amino acid (CXC-chemokines) or are adjacent (CC-chemokines). The CXC-chemokines include interleukin-8 (IL-8), neutrophil-activating protein-1 (NAP-1), neutrophil-activating protein-2 (NAP-2), GROα, GROβ, GROγ, ENA-78, GCP-2, IP-10, MIG and PF4. CC chemokines include RANTES, MIP-1α, MIP-2β, monocyte chemotactic protein-1 (MCP-1), MCP-2, MCP-3 and eotaxin. Individual members of the chemokine families are known to be bound by at least one chemokine receptor, with CXC-chemokines generally bound by members of the CXCR class of receptors, and CC-chemokines by members of the CCR class of receptors. For example, IL-8 is bound by the CXCR-1 and CXCR-2 receptors.

Since CXC-chemokines promote the accumulation and activation of neutrophils, these chemokines have been implicated in a wide range of acute and chronic inflammatory disorders including psoriasis and rheumatoid arthritis. Baggiolini et al., FEBS Lett. 307, 97 (1992); Miller et al., Crit. Rev. Immunol. 12, 17 (1992); Oppenheim et al., Annu. Fev. Immunol. 9, 617 (1991); Seitz et al., J. Clin. Invest. 87, 463 (1991); Miller et al., Am. Rev. Respir. Dis, 146, 427 (1992); Donnely et al., Lancet 341, 643 (1993).

ELRCXC chemokines including IL-8, GROα, GROβ, GROγ, NAP-2, and ENA-78 (Strieter et al. 1995 JBC 270 p. 27348-57) have also been implicated in the induction of tumor angiogenesis (new blood vessel growth). All of these chemokines are believed to exert their actions by binding to the 7 transmembrane G-protein coupled receptor CXCR2 (also known as IL-8RB), while IL-8 also binds CXCR1 (also known as IL-8RA). Thus, their angiogenic activity is due to their binding to and activation of CXCR2, and possible CXCR1 for IL-8, expressed on the surface of vascular endothelial cells (ECs) in surrounding vessels.

Many different types of tumors have been shown to produce ELRCXC chemokines and their production has been correlated with a more aggressive phenotype (Inoue et al. 2000 Clin Cancer Res 6 p. 2104-2119) and poor prognosis (Yoneda et. al. 1998 J Nat Cancer Inst 90 p. 447-454). Chemokines are potent chemotactic factors and the ELRCXC chemokines have been shown to induce EC chemotaxis. Thus, these chemokines probably induce chemotaxis of endothelial cells toward their site of production in the tumor. This may be a critical step in the induction of angiogenesis by the tumor. Inhibitors of CXCR2 or dual inhibitors of CXCR2 and CXCR1 will inhibit the angiogenic activity of the ELRCXC chemokines and therefore block the growth of the tumor. This anti-tumor activity has been demonstrated for antibodies to IL-8 (Arenberg et al. 1996 J Clin Invest 97 p. 2792-2802), ENA-78 (Arenberg et al. 1998 J Clin Invest 102 p. 465-72), and GROα (Haghnegahdar et al. J. Leukoc Biology 2000 67 p. 53-62).

Many tumor cells have also been shown to express CXCR2 and thus tumor cells may also stimulate their own growth when they secrete ELRCXC chemokines. Thus, along with decreasing angiogenesis, inhibitors of CXCR2 may directly inhibit the growth of tumor cells.

Hence, the CXC-chemokine receptors represent promising targets for the development of novel anti-inflammatory and anti-tumor agents.

There remains a need for compounds that are capable of modulating activity at CXC-chemokine receptors. For example, conditions associated with an increase in IL-8 production (which is responsible for chemotaxis of neutrophil and T-cell subsets into the inflammatory site and growth of tumors) would benefit by compounds that are inhibitors of IL-8 receptor binding.

### SUMMARY OF THE INVENTION

This invention provides compounds selected from the group consisting of the compounds of formulas 1 to 18 (as defined below).

This invention also provides pharmaceutically acceptable salts of the compounds selected from the group consisting of the compounds of formulas 1 to 18.

This invention also provides pharmaceutically acceptable esters of the compounds selected from the group consisting of the compounds of formulas 1 to 18.

This invention also provides solvates of the compounds selected from the group consisting of the compounds of formulas 1 to 18.

This invention also provides solvates of the compounds selected from the group consisting of the compounds of formulas 1 to 18, wherein said solvate is a hydrate (e.g., a monohydrate).

This invention also provides a method of treating, or inhibiting, a chemokine mediated disease in a patient in need of such treatment comprising administering to said patient an effective amount of at least one (e.g., 1, 2 or 3, or 1 or 2, or 1) compound selected from the group consisting of the compounds of formulas 1 to 18, the pharmaceutically acceptable salts thereof, the pharmaceutically acceptable esters thereof, and the solvates thereof.

This invention also provides a method of treating, or inhibiting, a chemokine mediated disease in a patient in need of such treatment comprising administering to said patient an effective amount a compound selected from the group consisting of the compounds of formulas 1 to 18, a pharmaceutically acceptable salt thereof, a pharmaceutically acceptable ester thereof, and a solvate thereof.

This invention also provides a method of treating, or inhibiting, a chemokine mediated disease in a patient in need of such treatment comprising administering to said patient an effective amount of at least one (e.g., 1, 2 or 3, or 1 or 2, or 1) compound selected from the group consisting of the compounds of formulas 1 to 18.

This invention also provides a method of treating, or inhibiting, a chemokine mediated disease in a patient in need of such treatment comprising administering to said patient an effective amount a compound selected from the group consisting of the compounds of formulas 1 to 18.

This invention also provides a method of treating, or inhibiting, a chemokine mediated disease in a patient in need of such treatment comprising administering to said patient an effective amount of a pharmaceutically acceptable salt of at least one (e.g., 1, 2 or 3, or 1 or 2, or 1) compound selected from the group consisting of the compounds of formulas 1 to 18.

This invention also provides a method of treating, or inhibiting, a chemokine mediated disease in a patient in need of such treatment comprising administering to said patient an effective amount a pharmaceutically acceptable salt of a compound selected from the group consisting of the compounds of formulas 1 to 18.

This invention also provides a method of treating, or inhibiting, a chemokine mediated disease in a patient in need of such treatment comprising administering to said patient an effective amount of a pharmaceutically acceptable ester of at least one (e.g., 1, 2 or 3, or 1 or 2, or 1) compound selected from the group consisting of the compounds of formulas 1 to 18.

This invention also provides a method of treating, or inhibiting, a chemokine mediated disease in a patient in need of such treatment comprising administering to said patient an effective amount a pharmaceutically acceptable ester of a compound selected from the group consisting of the compounds of formulas 1 to 18.

This invention also provides a method of treating, or inhibiting, a chemokine mediated disease in a patient in need of such treatment comprising administering to said patient an effective amount of a solvate of at least one (e.g., 1, 2 or 3, or 1 or 2, or 1) compound selected from the group consisting of the compounds of formulas 1 to 18.

This invention also provides a method of treating, or inhibiting, a chemokine mediated disease in a patient in need of such treatment comprising administering to said patient an effective amount a solvate of a compound selected from the group consisting of the compounds of formulas 1 to 18.

This invention also provides any one of the above methods of treating or inhibiting a chemokine mediated disease wherein the compound, pharmaceutically acceptable salt thereof, pharmaceutically acceptable ester thereof, or solvate thereof, is administered in combination (e.g., consecutively or sequentially) with one or more drugs, agents or therapeutics useful for the treatment of chemokine mediated diseases.

This invention also provides any one of the above methods of treating a chemokine mediated disease wherein said disease is cancer.

This invention also provides any one of the above methods of treating a chemokine mediated disease wherein said disease is cancer, and said compound (or salt thereof, or ester thereof, or solvate thereof) is administered concurrently or sequentially with: (a) a microtubule affecting agent, or (b) an antineoplastic agent, or (c) an anti-angiogenesis agent, or (d) a VEGF receptor kinase inhibitor, or (e) antibodies against the VEGF receptor, or (f) interferon, and/or (g) radiation.

This invention also provides any one of the above methods of inhibiting a chemokine mediated disease wherein said disease is angiogenesis.

This invention also provides any one of the above methods of treating a chemokine mediated disease wherein said disease is angiogenic ocular disease (e.g., ocular inflammation, retinopathy of prematurity, diabetic retinopathy, macular degeneration with the wet type preferred and corneal neovascularization).

This invention also provides any one of the above methods of treating a chemokine mediated disease wherein said disease is selected from the group consisting of: gingivitis, respiratory viruses, herpes viruses, hepatitis viruses, HIV, kaposi's sarcoma associated virus and atherosclerosis.

This invention also provides any one of the above methods of treating a chemokine mediated disease wherein said disease is acute inflammatory pain.

This invention also provides any one of the above methods of treating a chemokine mediated disease wherein said disease is chronic inflammatory pain.

This invention also provides any one of the above methods of treating a chemokine mediated disease wherein said disease is acute neuropathic pain.

This invention also provides any one of the above methods of treating a chemokine mediated disease wherein said disease is chronic neuropathic pain.

This invention also provides any one of the above methods of treating a chemokine mediated disease wherein said disease is COPD.

This invention also provides any one of the above methods of treating a chemokine mediated disease wherein said disease is psoriasis.

This invention also provides any one of the above methods of treating a chemokine mediated disease wherein said disease is Asthma.

This invention also provides any one of the above methods of treating a chemokine mediated disease wherein said disease is acute inflammation.

This invention also provides any one of the above methods of treating a chemokine mediated disease wherein said disease is chronic inflammation.

This invention also provides any one of the above methods of treating a chemokine mediated disease wherein said disease is rheumatoid arthritis.

This invention also provides a pharmaceutical composition comprising an effective amount of at least one (e.g., 1-3, usually 1) compound selected from the group of consisting of the compounds of formulas 1 to 18.

This invention also provides a pharmaceutical composition comprising an is effective amount of at least one (e.g., 1-3, usually 1) pharmaceutically acceptable salt of at least one (e.g., 1-3, usually 1) compound selected from the group of consisting of the compounds of formulas 1 to 18.

This invention also provides a pharmaceutical composition comprising an effective amount a pharmaceutically acceptable salt of a compound selected from the group of consisting of the compounds of formulas 1 to 18.

This invention also provides a pharmaceutical composition comprising an effective amount of at least one (e.g., 1-3, usually 1) pharmaceutically acceptable ester of at least one (e.g., 1-3, usually 1) compound selected from the group of consisting of the compounds of formulas 1 to 18.

This invention also provides a pharmaceutical composition comprising an effective amount of a pharmaceutically acceptable ester of a compound selected from the group of consisting of the compounds of formulas 1 to 18.

This invention also provides a pharmaceutical composition comprising an effective amount of at least one (e.g., 1-3, usually 1) solvate of at least one (e.g., 1-3, usually 1) compound selected from the group of consisting of the compounds of formulas 1 to 18.

This invention also provides a pharmaceutical composition comprising an effective amount of a solvate of a compound selected from the group of consisting of the compounds of formulas 1 to 18.

This invention also provides any of the above methods of treating, or inhibiting, chemokine mediated diseases wherein any one of the above pharmaceutical compositions is administered to the patient in need of treatment.

This invention also provides prodrugs of the compounds of formulas 1 to 18.

This invention also provides any of the compounds of formulas 1 to 18 in isolated form.

This invention also provides any of the compounds of formulas 1 to 18 in pure form.

This invention also provides any of the compounds of formulas 1 to 18 in pure and isolated form.

### DETAILED DESCRIPTION OF THE INVENTION

Unless indicated otherwise, the following definitions apply throughout the present specification and claims. These definitions apply regardless of whether a term is used by itself or in combination with other terms.

“At least one” represents, for example, 1, or 1 or 2, or 1, 2 or 3.

“One or more” represents, for example, 1, 1 or 2, or 1, 2 or 3.

“Patient” includes both human and other mammals, preferably human.

“Mammal” includes a human being, and preferably means a human being.

The term “prodrug,” as used herein, represents compounds which are rapidly transformed in vivo to the parent compound of the above formula, for example, by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As used herein, the term “composition” is intended to encompass a product comprising the specified ingredients in the specified amounts, as well as any product which results, directly or indirectly, from combination of the specified ingredients in the specified amounts.

As used in the methods of this invention, “an effective amount” means a therapeutically acceptable amount (i.e., that amount which provides the desired therapeutic effective).

The compounds of formulas 1 to 18, i.e., the compounds of this invention, are:

| Structure | CHEMICAL NAME |
|---|---|
| 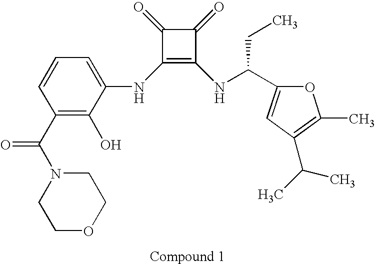 | 4-[2-HYDROXY-3-[[2-[[1(R)-[5-METHYL- 4-(1-METHYLETHYL)-2-FURANYL]- PROPYL]AMINO]-3,4-DIOXO-1- CYCLOBUTEN-1-YL]AMINO]BENZOYL]- MORPHOLINE |
| 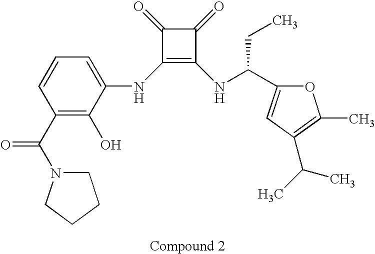 | 1-[2-HYDROXY-3-[[2-[[1(R)-[5-METHYL- 4-(1-METHYLETHYL)-2-FURANLY]- PROPYL]AMINO]-3,4-DIOXO-1- CYCLOBUTEN-1-YL]AMINO]-BENZOYL]PYRROLIDINE |
| 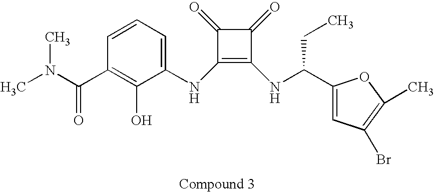 | 3-[[2-[[1(R)-(4-BROMO-5-METHYL-2- FURANYL)PROPYL]AMINO]-3,4-DIOXO- 1-CYCLOBUTEN-1-YL]AMINO]-2- HYDROXY-N,N-DIMETHYLBENZAMIDE |
| 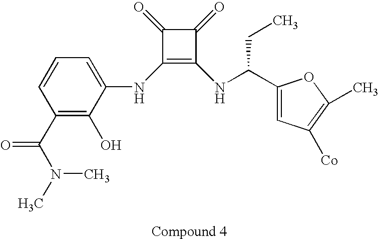 | 3-[[2-[[1(R)-(4-CHLORO-5-METHYL-2- FURANYL)PROPYL]AMINO]-3,4-DIOXO- 1-CYCLOBUTEN-1-YL]AMINO]-2- HYDROXY-N,N-DIMETHYLBENZAMIDE |
| 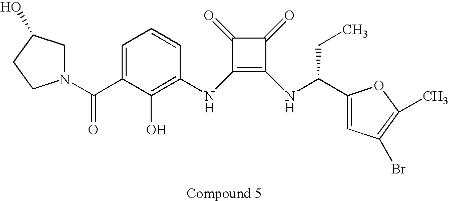 | 1-[3-[[2-[[1(R)-(4-BROMO-5-METHYL-2- FURANYL)PROPYL]AMINO]-3,4-DIOXO- 1-CYCLOBUTEN-1-YL]AMINO]-2- HYDROXYBENZOYL]-3(S)- PYRROLIDINOL |
| 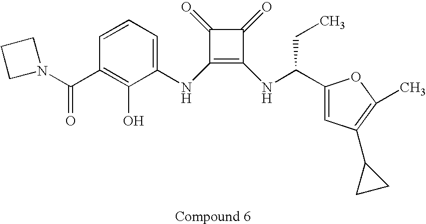 | 1-[3-[[2-[[1(R)-(4-CYCLOPROPYL-5- METHYL-2-FURANYL)PROPYL]AMINO]- 3,4-DIOXO-1-CYCLOBUTEN-1- YL]AMINO]-2-HYDROXYBENZOYL]- AZETIDINE |
| 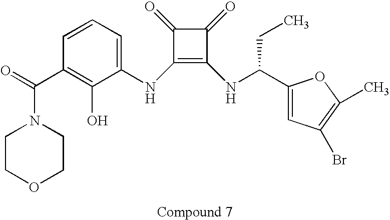 | 4-[3-[[2-[[1(R)-(4-BROMO-5-METHYL-2- FURANYL)PROPYL]AMINO]-3,4-DIOXO- 1-CYCLOBUTEN-1-YL]AMINO]-2- HYDROXYBENZOYL]MORPHOLINE |
| 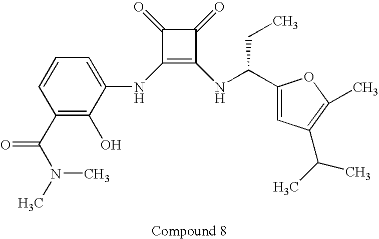 | 2-HYDROXY-N,N-DIMETHYL-3-[[2- [[1(R)-[5-METHYL-4-(1-METHYLETHYL)- 2-FURANYL]PROPYL]AMINO]-3,4- DIOXO-1-CYCLOBUTEN-1-YL]AMINO]- BENZAMIDE |
| 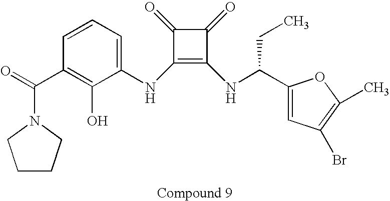 | 1-[3-[[2-[[1(R)-(4-BROMO-5-METHYL-2- FURANYL)PROPYL]AMINO]-3,4-DIOXO- 1-CYCLOBUTEN-1-YL]AMINO]-2- HYDROXYBENZOYL]PYRROLIDINE |
| 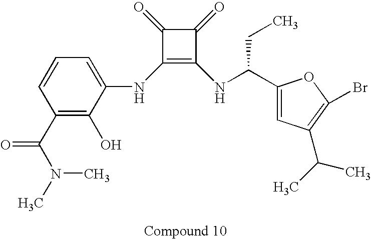 | 3-[[2-[[1(R)-[5-BROMO-4-(1-METHYL- ETHYL)-2-FURANYL]PROPYL]AMINO]- 3,4,-DIOXO-1-CYCLOBUTEN-1-YL]- AMINO]-2-HYDROXY-N,N-DIMETHYL- BENZAMIDE |
| 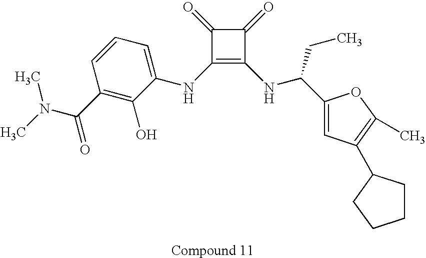 | 3-[[2-[[1(R)-(4-CYCLOPENTYL-5- METHYL-2-FURANYL)PROPYL]AMINO]- 3,4-DIOXO-1-CYCLOBUTEN-1-YL]- AMINO]-2-HYDROXY-N,N-DIMETHYL- BENZAMIDE |
| 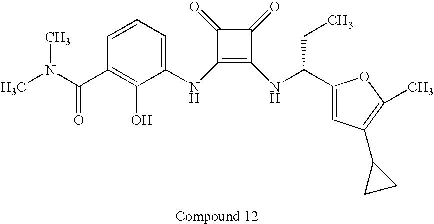 | 3-[[2-[[1(R)-(4-CYCLOPROPYL-5- METHYL-2-FURANYL)PROPYL]AMINO]- 3,4-DIOXO-1-CYCLOBUTEN-1-YL]- AMINO]-2-HYDROXY-N,N-DIMETHYL- BENZAMIDE |
| 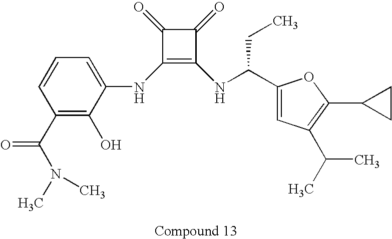 | 3-[[2-[[1(R)-[5-CYCLOPROPYL-4-(1- METHYLETHYL)-2-FURANYL]PROPYL]- AMINO]-3,4-DIOXO-1-CYCLOBUTEN-1- YL]AMINO]-2-HYDROXY-N,N- DIMETHYLBENZAMIDE |
| 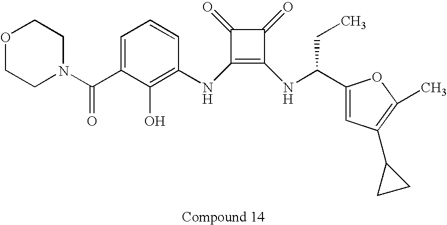 | 4-[3-[[2-[[1(R)-(4-CYCLOPROPYL-5- METHYL-2-FURANYL)PROPYL]AMINO]- 3,4-DIOXO-1-CYCLOBUTEN-1-YL]- AMINO]-2-HYDROXYBENZOYL]- MORPHOLINE |
| 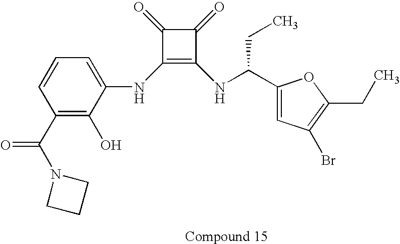 | 1-[3-[[2-[[1(R)-(4-BROMO-5-ETHYL-2- FURANYL)PROPYL]AMINO]-3,4-DIOXO- 1-CYCLOBUTEN-1-YL]AMINO]-2- HYDROXYBENZOYL]AZETIDINE |
| 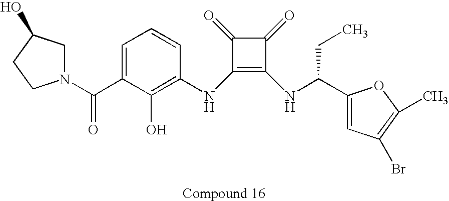 | 1-[3-[[2-[[1(R)-(4-BROMO-5-METHYL-2- FURANYL)PROPYL]AMINO]-3,4-DIOXO- 1-CYCLOBUTEN-1-YL]AMINO]-2- HYDROXYBENZOYL]-3(R)- PYRROLIDINOL |
| 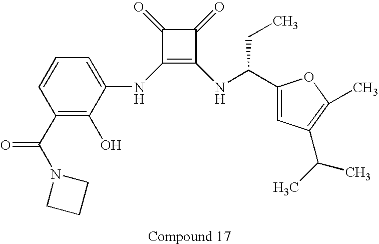 | 1-[2-HYDROXY-3-[[2-[[1(R)-[5-METHYL- 4-(1-METHYLETHYL)-2-FURANYL]- PROPYL]AMINO]-3,4-DIOXO-1- CYCLOBUTEN-1-YL]AMINO]- BENZOYL]AZETIDINE |
| 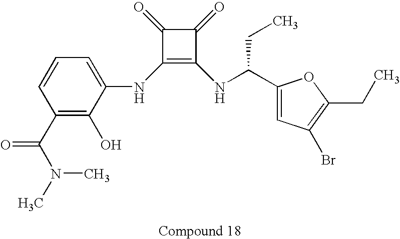 | 3-[[2-[[1(R)-(4-BROMO-5-ETHYL-2- FURANYL)PROPYL]AMINO]-3,4-DIOXO- 1-CYCLOBUTEN-1-YL]AMINO]-2- HYDROXY-N,N-DIMETHYLBENZAMIDE |

Another embodiment of the present invention is directed to a method of treating a chemokine mediated disease in a patient in need of such treatment (e.g., a mammal, preferably a human being) comprising administering to said patient a therapeutically effective amount of at least one (e.g., 1-3, and usually one) compound selected from the group consisting of compounds of formulas 1 to 18, the pharmaceutically acceptable salts thereof, the esters thereof, and the solvates thereof.

Examples of chemokine mediated diseases include: acute inflammation, chronic inflammation, rheumatoid arthritis, acute inflammatory pain, chronic to inflammatory pain, acute neuropathic pain, chronic neuropathic pain, psoriasis, atopic dermatitis, asthma, COPD, adult respiratory disease, arthritis, inflammatory bowel disease, Crohn's disease, ulcerative colitis, septic shock, endotoxic shock, gram negative sepsis, toxic shock syndrome, stroke, cardiac and renal reperfusion injury, glomerulonephritis, thrombosis, Alzheimer's disease, graft vs. host reaction, allograft rejections, malaria, acute respiratory distress syndrome, delayed type hypersensitivity reaction, atherosclerosis, cerebral and cardiac ischemia, osteoarthritis, multiple sclerosis, restinosis, angiogenesis, osteoporosis, gingivitis, respiratory viruses, herpes viruses, hepatitis viruses, HIV, Kaposi's sarcoma associated virus, meningitis, cystic fibrosis, pre-term labor, cough, pruritis, multi-organ dysfunction, trauma, strains, sprains, contusions, psoriatic arthritis, herpes, encephalitis, CNS vasculitis, traumatic to brain injury, CNS tumors, subarachnoid hemorrhage, post surgical trauma, interstitial pneumonitis, hypersensitivity, crystal induced arthritis, acute and chronic pancreatitis, acute alcoholic hepatitis, necrotizing enterocolitis, chronic sinusitis, angiogenic ocular disease, ocular inflammation, retinopathy of prematurity, diabetic retinopathy, macular degeneration with the wet type preferred and corneal neovascularization, polymyositis, vasculitis, acne, gastric and duodenal ulcers, celiac disease, esophagitis, glossitis, airflow obstruction, airway hyperresponsiveness, bronchiectasis, bronchiolitis, bronchiolitis obliterans, chronic bronchitis, cor pulmonae, cough, dyspnea, emphysema, hypercapnea, hyperinflation, hypoxemia, hyperoxia-induced inflammations, hypoxia, surgical lung volume reduction, pulmonary fibrosis, pulmonary hypertension, right ventricular hypertrophy, peritonitis associated with continuous ambulatory peritoneal dialysis (CAPD), granulocytic ehrlichiosis, sarcoidosis, small airway disease, ventilation-perfusion mismatching, wheeze, colds, gout, alcoholic liver disease, lupus, burn therapy, periodontitis, transplant reperfusion injury and early transplantation rejection.

An embodiment of the present invention is directed to a method of treating cancer in a patient (e.g., a mammal, such as a human being) in need of such treatment, comprising administering to said patient, concurrently or sequentially, a therapeutically effective amount of (a) at least one (e.g., 1-3, and usually one) compound selected from the group consisting of the compounds of formulas 1 to 18 (or pharmaceutically acceptable salts, esters or solvates thereof), and (b) a microtubule affecting agent or antineoplastic agent or anti-angiogenesis agent or VEGF receptor kinase inhibitor or antibodies against the VEGF receptor or interferon, and/or c) radiation.

In another embodiment directed to the treatment of cancer, at least one (e.g., 1-3, and usually one) compound, selected from the group consisting of compounds of formulas 1 to 18 (or pharmaceutically acceptable salts, esters or solvates thereof), is administered in combination with antineoplastic agents (e.g., one or more, such as one, or such as one or two), selected from the group consisting of: gemcitabine, paclitaxel (Taxol®), 5-Fluorouracil (5-FU), cyclophosphamide (Cytoxan®), temozolomide, taxotere and Vincristine.

In another embodiment the present invention provides a method of treating cancer in a patient (e.g., a mammal, such as a human being) in need of such treatment, comprising administering, concurrently or sequentially, an effective amount of (a) a compound selected from the group consisting of compounds of formulas 1 to 18 (or pharmaceutically acceptable salts, esters or solvates thereof), and (b) a microtubule affecting agent (e.g., paclitaxel).

In another embodiment the present invention provides a method of treating cancer in a patient (e.g., a mammal, such as a human being) in need of such treatment, comprising administering, concurrently or sequentially, an effective amount of (a) a compound selected from the group consisting of compounds of formulas 1 to 18 (or pharmaceutically acceptable salts, esters or solvates thereof), and (b) an antineoplastic agent, microtubule affecting agent or anti-angiogenesis agent.

Another embodiment of the present invention is directed to a method of treating acute inflammatory pain in a patient in need of such treatment (e.g., a mammal, preferably a human being) comprising administering to said patient a therapeutically effective amount of at least one (e.g., 1-3, and usually one) compound selected from the group consisting of compounds of formulas 1 to 18 (or pharmaceutically acceptable salts, esters or solvates thereof).

Another embodiment of the present invention is directed to a method of treating chronic inflammatory pain in a patient in need of such treatment (e.g., a mammal, preferably a human being) comprising administering to said patient a therapeutically effective amount of at least one (e.g., 1-3, and usually one) compound selected from the group consisting of compounds of formulas 1 to 18 (or pharmaceutically acceptable salts, esters or solvates thereof).

Another embodiment of the present invention is directed to a method of treating acute neuropathic pain in a patient in need of such treatment (e.g., a mammal, preferably a human being) comprising administering to said patient a therapeutically effective amount of at least one (e.g., 1-3, and usually one) compound selected from the group consisting of compounds of formulas 1 to 18 (or pharmaceutically acceptable salts, esters or solvates thereof).

Another embodiment of the present invention is directed to a method of treating chronic neuropathic pain in a patient in need of such treatment (e.g., a mammal, preferably a human being) comprising administering to said patient a therapeutically effective amount of at least one (e.g., 1-3, and usually one) compound selected from the group consisting of compounds of formulas 1 to 18 (or pharmaceutically acceptable salts, esters or solvates thereof).

Another embodiment of the present invention is directed to a method of treating COPD in a patient in need of such treatment (e.g., a mammal, preferably a human being) comprising administering to said patient a therapeutically effective amount of at least one (e.g., 1-3, and usually one) compound selected from the group consisting of compounds of formulas 1 to 18 (or pharmaceutically acceptable salts, esters or solvates thereof).

Another embodiment of the present invention is directed to a method of treating COPD in a patient in need of such treatment (e.g., a mammal, preferably a human being) comprising administering to said patient a therapeutically effective amount of a compound selected from the group consisting of compounds of formulas 1 to 18.

Another embodiment of the present invention is directed to a method of treating COPD in a patient in need of such treatment (e.g., a mammal, preferably a human being) comprising administering to said patient a therapeutically effective amount of a solvate of a compound selected from the group consisting of compounds of formulas 1 to 18.

Another embodiment of the present invention is directed to a method of treating COPD in a patient in need of such treatment (e.g., a mammal, preferably a human being) comprising administering to said patient a therapeutically effective amount of a solvate of a compound selected from the group consisting of compounds of formulas 1 to 18, wherein said solvate is a hydrate (e.g., a monohydrate).

Another embodiment of the present invention is directed to a method of treating COPD in a patient in need of such treatment (e.g., a mammal, preferably a human being) comprising administering to said patient a therapeutically effective amount of a compound of formula 1.

Another embodiment of the present invention is directed to a method of treating COPD in a patient in need of such treatment (e.g., a mammal, preferably a human being) comprising administering to said patient a therapeutically effective amount of a solvate of a compound of formula 1.

Another embodiment of the present invention is directed to a method of treating COPD in a patient in need of such treatment (e.g., a mammal, preferably a human being) comprising administering to said patient a therapeutically effective amount of a in solvate of a compound of formula 1, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 1, wherein said solvate is a monohydrate.

Another embodiment of the present invention is directed to a method of treating COPD in a patient in need of such treatment (e.g., a mammal, preferably a human being) comprising administering to said patient a therapeutically effective amount of a compound of formula 2.

Another embodiment of the present invention is directed to a method of treating COPD in a patient in need of such treatment (e.g., a mammal, preferably a human being) comprising administering to said patient a therapeutically effective amount of a solvate of a compound of formula 2.

Another embodiment of the present invention is directed to a method of treating COPD in a patient in need of such treatment (e.g., a mammal, preferably a human being) comprising administering to said patient a therapeutically effective amount of a solvate of a compound of formula 2, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 2, wherein said solvate is a monohydrate.

Another embodiment of the present invention is directed to a method of treating COPD in a patient in need of such treatment (e.g., a mammal, preferably a human being) comprising administering to said patient a therapeutically effective amount of a compound of formula 3.

Another embodiment of the present invention is directed to a method of treating COPD in a patient in need of such treatment (e.g., a mammal, preferably a human being) comprising administering to said patient a therapeutically effective amount of a solvate of a compound of formula 3.

Another embodiment of the present invention is directed to a method of treating COPD in a patient in need of such treatment (e.g., a mammal, preferably a human being) comprising administering to said patient a therapeutically effective amount of a solvate of a compound of formula 3, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 3, wherein said solvate is a monohydrate.

Another embodiment of the present invention is directed to a method of treating COPD in a patient in need of such treatment (e.g., a mammal, preferably a human being) comprising administering to said patient a therapeutically effective amount of a compound of formula 4.

Another embodiment of the present invention is directed to a method of treating COPD in a patient in need of such treatment (e.g., a mammal, preferably a human being) comprising administering to said patient a therapeutically effective amount of a solvate of a compound of formula 4.

Another embodiment of the present invention is directed to a method of treating COPD in a patient in need of such treatment (e.g., a mammal, preferably a human being) comprising administering to said patient a therapeutically effective amount of a solvate of a compound of formula 4, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 4, wherein said solvate is a monohydrate.

Another embodiment of the present invention is directed to a method of treating COPD in a patient in need of such treatment (e.g., a mammal, preferably a human being) comprising administering to said patient a therapeutically effective amount of a compound of formula 5.

Another embodiment of the present invention is directed to a method of treating COPD in a patient in need of such treatment (e.g., a mammal, preferably a human being) comprising administering to said patient a therapeutically effective amount of a solvate of a compound of formula 5.

Another embodiment of the present invention is directed to a method of treating COPD in a patient in need of such treatment (e.g., a mammal, preferably a human being) comprising administering to said patient a therapeutically effective amount of a solvate of a compound of formula 5, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 5, wherein said solvate is a monohydrate.

Another embodiment of the present invention is directed to a method of treating COPD in a patient in need of such treatment (e.g., a mammal, preferably a human being) comprising administering to said patient a therapeutically effective amount of a compound of formula 6.

Another embodiment of the present invention is directed to a method of treating COPD in a patient in need of such treatment (e.g., a mammal, preferably a human being) comprising administering to said patient a therapeutically effective amount of a solvate of a compound of formula 6.

Another embodiment of the present invention is directed to a method of treating COPD in a patient in need of such treatment (e.g., a mammal, preferably a human being) comprising administering to said patient a therapeutically effective amount of a solvate of a compound of formula 6, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 6, wherein said solvate is a monohydrate.

Another embodiment of the present invention is directed to a method of treating COPD in a patient in need of such treatment (e.g., a mammal, preferably a human being) comprising administering to said patient a therapeutically effective amount of a compound of formula 7.

Another embodiment of the present invention is directed to a method of treating COPD in a patient in need of such treatment (e.g., a mammal, preferably a human being) comprising administering to said patient a therapeutically effective amount of a solvate of a compound of formula 7.

Another embodiment of the present invention is directed to a method of treating COPD in a patient in need of such treatment (e.g., a mammal, preferably a human being) comprising administering to said patient a therapeutically effective amount of a solvate of a compound of formula 7, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 7, wherein said solvate is a monohydrate.

Another embodiment of the present invention is directed to a method of treating COPD in a patient in need of such treatment (e.g., a mammal, preferably a human being) comprising administering to said patient a therapeutically effective amount of a compound of formula 8.

Another embodiment of the present invention is directed to a method of treating COPD in a patient in need of such treatment (e.g., a mammal, preferably a human being) comprising administering to said patient a therapeutically effective amount of a solvate of a compound of formula 8.

Another embodiment of the present invention is directed to a method of treating COPD in a patient in need of such treatment (e.g., a mammal, preferably a human being) comprising administering to said patient a therapeutically effective amount of a solvate of a compound of formula 8, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 8, wherein said solvate is a monohydrate.

Another embodiment of the present invention is directed to a method of treating COPD in a patient in need of such treatment (e.g., a mammal preferably a human being) comprising administering to said patient a therapeutically effective amount of a compound of formula 9.

Another embodiment of the present invention is directed to a method of treating COPD in a patient in need of such treatment (e.g., a mammal, preferably a human being) comprising administering to said patient a therapeutically effective amount of a solvate of a compound of formula 9.

Another embodiment of the present invention is directed to a method of treating COPD in a patient in need of such treatment (e.g., a mammal, preferably a human being) comprising administering to said patient a therapeutically effective amount of a solvate of a compound of formula 9, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 9, wherein said solvate is a monohydrate.

Another embodiment of the present invention is directed to a method of treating COPD in a patient in need of such treatment (e.g., a mammal, preferably a human being) comprising administering to said patient a therapeutically effective amount of a compound of formula 10.

Another embodiment of the present invention is directed to a method of treating COPD in a patient in need of such treatment (e.g., a mammal, preferably a human being) comprising administering to said patient a therapeutically effective amount of a solvate of a compound of formula 10.

Another embodiment of the present invention is directed to a method of treating COPD in a patient in need of such treatment (e.g., a mammal, preferably a human being) comprising administering to said patient a therapeutically effective amount of a solvate of a compound of formula 10, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 10, wherein said solvate is a monohydrate.

Another embodiment of the present invention is directed to a method of treating COPD in a patient in need of such treatment (e.g., a mammal, preferably a human being) comprising administering to said patient a therapeutically effective amount of a compound of formula 1.

Another embodiment of the present invention is directed to a method of treating COPD in a patient in need of such treatment (e.g., a mammal, preferably a human being) comprising administering to said patient a therapeutically effective amount of a solvate of a compound of formula 11.

Another embodiment of the present invention is directed to a method of treating COPD in a patient in need of such treatment (e.g., a mammal, preferably a human being) comprising administering to said patient a therapeutically effective amount of a solvate of a compound of formula 11, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 11, wherein said solvate is a monohydrate.

Another embodiment of the present invention is directed to a method of treating COPD in a patient in need of such treatment (e.g., a mammal, preferably a human being) comprising administering to said patient a therapeutically effective amount of a compound of formula 12.

Another embodiment of the present invention is directed to a method of treating COPD in a patient in need of such treatment (e.g., a mammal, preferably a human being) comprising administering to said patient a therapeutically effective amount of a solvate of a compound of formula 12.

Another embodiment of the present invention is directed to a method of treating COPD in a patient in need of such treatment (e.g., a mammal, preferably a human being) comprising administering to said patient a therapeutically effective amount of a solvate of a compound of formula 12, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 12, wherein said solvate is a monohydrate.

Another embodiment of the present invention is directed to a method of treating COPD in a patient in need of such treatment (e.g., a mammal, preferably a human being) comprising administering to said patient a therapeutically effective amount of a compound of formula 13.

Another embodiment of the present invention is directed to a method of treating COPD in a patient in need of such treatment (e.g., a mammal, preferably a human being) comprising administering to said patient a therapeutically effective amount of a solvate of a compound of formula 13.

Another embodiment of the present invention is directed to a method of treating COPD in a patient in need of such treatment (e.g., a mammal, preferably a human being) comprising administering to said patient a therapeutically effective amount of a solvate of a compound of formula 13, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 13, wherein said solvate is a monohydrate.

Another embodiment of the present invention is directed to a method of treating COPD in a patient in need of such treatment (e.g., a mammal, preferably a human being) comprising administering to said patient a therapeutically effective amount of a compound of formula 14.

Another embodiment of the present invention is directed to a method of treating COPD in a patient in need of such treatment (e.g., a mammal, preferably a human being) comprising administering to said patient a therapeutically effective amount of a solvate of a compound of formula 14.

Another embodiment of the present invention is directed to a method of treating COPD in a patient in need of such treatment (e.g., a mammal, preferably a human being) comprising administering to said patient a therapeutically effective amount of a solvate of a compound of formula 14, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 14, wherein said solvate is a monohydrate.

Another embodiment of the present invention is directed to a method of treating COPD in a patient in need of such treatment (e.g., a mammal, preferably a human being) comprising administering to said patient a therapeutically effective amount of a compound of formula 15.

Another embodiment of the present invention is directed to a method of treating COPD in a patient in need of such treatment (e.g., a mammal, preferably a human being) comprising administering to said patient a therapeutically effective amount of a solvate of a compound of formula 15.

Another embodiment of the present invention is directed to a method of treating COPD in a patient in need of such treatment (e.g., a mammal, preferably a human being) comprising administering to said patient a therapeutically effective amount of a solvate of a compound of formula 15, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 15, wherein said solvate is a monohydrate.

Another embodiment of the present invention is directed to a method of treating COPD in a patient in need of such treatment (e.g., a mammal, preferably a human being) comprising administering to said patient a therapeutically effective amount of a compound of formula 16.

Another embodiment of the present invention is directed to a method of treating COPD in a patient in need of such treatment (e.g., a mammal, preferably a human being) comprising administering to said patient a therapeutically effective amount of a solvate of a compound of formula 16.

Another embodiment of the present invention is directed to a method of treating COPD in a patient in need of such treatment (e.g., a mammal, preferably a human being) comprising administering to said patient a therapeutically effective amount of a solvate of a compound of formula 16, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 16, wherein said solvate is a monohydrate.

Another embodiment of the present invention is directed to a method of treating COPD in a patient in need of such treatment (e.g., a mammal, preferably a human being) comprising administering to said patient a therapeutically effective amount of a compound of formula 17.

Another embodiment of the present invention is directed to a method of treating COPD in a patient in need of such treatment (e.g., a mammal, preferably a human being) comprising administering to said patient a therapeutically effective amount of a solvate of a compound of formula 17.

Another embodiment of the present invention is directed to a method of treating COPD in a patient in need of such treatment (e.g., a mammal, preferably a human being) comprising administering to said patient a therapeutically effective amount of a to solvate of a compound of formula 17, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 17, wherein said solvate is a monohydrate. Another embodiment of the present invention is directed to a method of treating COPD in a patient in need of such treatment (e.g., a mammal, preferably a human being) comprising administering to said patient a therapeutically effective amount of a compound of formula 18.

Another embodiment of the present invention is directed to a method of treating COPD in a patient in need of such treatment (e.g., a mammal, preferably a human being) comprising administering to said patient a therapeutically effective amount of a solvate of a compound of formula 18.

Another embodiment of the present invention is directed to a method of treating COPD in a patient in need of such treatment (e.g., a mammal, preferably a human being) comprising administering to said patient a therapeutically effective amount of a solvate of a compound of formula 18, wherein said solvate is a hydrate.

Another embodiment of the present invention is directed to a method of treating COPD in a patient in need of such treatment (e.g., a mammal, preferably a human being) comprising administering to said patient a therapeutically effective amount of a solvate of a compound of formula 18, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of at least one (e.g., 1-3, and usually one) compound selected from the group consisting of compounds of formulas 1 to 18 (or pharmaceutically acceptable salts, esters or solvates thereof).

Another embodiment of the present invention is directed to a method of treating psoriasis in a patient in need of such treatment (e.g., a mammal, preferably a human being) comprising administering to said patient a therapeutically effective amount of a compound selected from the group consisting of compounds of formulas 1 to 18.

Another embodiment of the present invention is directed to a method of treating psoriasis in a patient in need of such treatment (e.g., a mammal, preferably a human being) comprising administering to said patient a therapeutically effective amount of a solvate of a compound selected from the group consisting of compounds of formulas 1 to 18.

Another embodiment of the present invention is directed to a method of treating psoriasis in a patient in need of such treatment (e.g., a mammal, preferably a human being) comprising administering to said patient a therapeutically effective amount of a solvate of a compound selected from the group consisting of compounds of formulas 1 to 18, wherein said solvate is a hydrate (e.g., a monohydrate).

Another embodiment of the present invention is directed to a method of treating psoriasis in a patient in need of such treatment (e.g., a mammal, preferably a human being) comprising administering to said patient a therapeutically effective amount of a compound of formula 1.

Another embodiment of the present invention is directed to a method of treating psoriasis in a patient in need of such treatment (e.g., a mammal, preferably a human being) comprising administering to said patient a therapeutically effective amount of a solvate of a compound of formula 1.

Another embodiment of the present invention is directed to a method of treating psoriasis in a patient in need of such treatment (e.g., a mammal, preferably a human being) comprising administering to said patient a therapeutically effective amount of a solvate of a compound of formula 1, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 1, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 2.

Another embodiment of the present invention is directed to a method of treating psoriasis in a patient in need of such treatment (e.g., a mammal, preferably a human being) comprising administering to said patient a therapeutically effective amount of a solvate of a compound of formula 2.

Another embodiment of the present invention is directed to a method of treating psoriasis in a patient in need of such treatment (e.g., a mammal, preferably a human being) comprising administering to said patient a therapeutically effective amount of a solvate of a compound of formula 2, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 2, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 3.

Another embodiment of the present invention is directed to a method of treating psoriasis in a patient in need of such treatment (e.g., a mammal, preferably a human being) comprising administering to said patient a therapeutically effective amount of a solvate of a compound of formula 3.

Another embodiment of the present invention is directed to a method of treating psoriasis in a patient in need of such treatment (e.g., a mammal, preferably a human being) comprising administering to said patient a therapeutically effective amount of a solvate of a compound of formula 3, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 3, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 4.

Another embodiment of the present invention is directed to a method of treating psoriasis in a patient in need of such treatment (e.g., a mammal, preferably a human being) comprising administering to said patient a therapeutically effective amount of a solvate of a compound of formula 4.

Another embodiment of the present invention is directed to a method of treating psoriasis in a patient in need of such treatment (e.g., a mammal, preferably a human being) comprising administering to said patient a therapeutically effective amount of a solvate of a compound of formula 4, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 4, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 5.

Another embodiment of the present invention is directed to a method of treating psoriasis in a patient in need of such treatment (e.g., a mammal, preferably a human being) comprising administering to said patient a therapeutically effective amount of a solvate of a compound of formula 5.

Another embodiment of the present invention is directed to a method of treating psoriasis in a patient in need of such treatment (e.g., a mammal, preferably a human being) comprising administering to said patient a therapeutically effective amount of a solvate of a compound of formula 5, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 5, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 6.

Another embodiment of the present invention is directed to a method of treating psoriasis in a patient in need of such treatment (e.g., a mammal, preferably a human being) comprising administering to said patient a therapeutically effective amount of a solvate of a compound of formula 6.

Another embodiment of the present invention is directed to a method of treating psoriasis in a patient in need of such treatment (e.g., a mammal, preferably a human being) comprising administering to said patient a therapeutically effective amount of a solvate of a compound of formula 6, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 6, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 7.

Another embodiment of the present invention is directed to a method of treating psoriasis in a patient in need of such treatment (e.g., a mammal, preferably a human being) comprising administering to said patient a therapeutically effective amount of a solvate of a compound of formula 7.

Another embodiment of the present invention is directed to a method of treating psoriasis in a patient in need of such treatment (e.g., a mammal, preferably a human being) comprising administering to said patient a therapeutically effective amount of a solvate of a compound of formula 7, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 7, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 8.

Another embodiment of the present invention is directed to a method of treating psoriasis in a patient in need of such treatment (e.g., a mammal, preferably a human being) comprising administering to said patient a therapeutically effective amount of a solvate of a compound of formula 8.

Another embodiment of the present invention is directed to a method of treating psoriasis in a patient in need of such treatment (e.g., a mammal, preferably a human being) comprising administering to said patient a therapeutically effective amount of a solvate of a compound of formula 8, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 8, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 9.

Another embodiment of the present invention is directed to a method of treating psoriasis in a patient in need of such treatment (e.g., a mammal, preferably a human being) comprising administering to said patient a therapeutically effective amount of a solvate of a compound of formula 9.

Another embodiment of the present invention is directed to a method of treating psoriasis in a patient in need of such treatment (e.g., a mammal, preferably a human being) comprising administering to said patient a therapeutically effective amount of a solvate of a compound of formula 9, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 9, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 10.

Another embodiment of the present invention is directed to a method of treating psoriasis in a patient in need of such treatment (e.g., a mammal, preferably a human being) comprising administering to said patient a therapeutically effective amount of a solvate of a compound of formula 10.

Another embodiment of the present invention is directed to a method of treating psoriasis in a patient in need of such treatment (e.g., a mammal, preferably a human being) comprising administering to said patient a therapeutically effective amount of a solvate of a compound of formula 10, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 10, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 11.

Another embodiment of the present invention is directed to a method of treating psoriasis in a patient in need of such treatment (e.g., a mammal, preferably a human being) comprising administering to said patient a therapeutically effective amount of a solvate of a compound of formula 11.

Another embodiment of the present invention is directed to a method of treating psoriasis in a patient in need of such treatment (e.g., a mammal, preferably a human being) comprising administering to said patient a therapeutically effective amount of a solvate of a compound of formula 11, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 11, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 12.

Another embodiment of the present invention is directed to a method of treating psoriasis in a patient in need of such treatment (e.g., a mammal, preferably a human being) comprising administering to said patient a therapeutically effective amount of a solvate of a compound of formula 12.

Another embodiment of the present invention is directed to a method of treating psoriasis in a patient in need of such treatment (e.g., a mammal, preferably a human being) comprising administering to said patient a therapeutically effective amount of a solvate of a compound of formula 12, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 12, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 13.

Another embodiment of the present invention is directed to a method of treating psoriasis in a patient in need of such treatment (e.g., a mammal, preferably a human being) comprising administering to said patient a therapeutically effective amount of a solvate of a compound of formula 13.

Another embodiment of the present invention is directed to a method of treating psoriasis in a patient in need of such treatment (e.g., a mammal, preferably a human being) comprising administering to said patient a therapeutically effective amount of a solvate of a compound of formula 13, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 13, wherein said solvate is a monohydrate.

Another embodiment of the present invention is directed to a method of treating psoriasis in a patient in need of such treatment (e.g., a mammals preferably a human being) comprising administering to said patient a therapeutically effective amount of a compound of formula 14.

Another embodiment of the present invention is directed to a method of treating psoriasis in a patient in need of such treatment (e.g., a mammal, preferably a human being) comprising administering to said patient a therapeutically effective amount of a solvate of a compound of formula 14.

Another embodiment of the present invention is directed to a method of treating psoriasis in a patient in need of such treatment (e.g., a mammal, preferably a human being) comprising administering to said patient a therapeutically effective amount of a solvate of a compound of formula 14, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 14, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 15.

Another embodiment of the present invention is directed to a method of treating psoriasis in a patient in need of such treatment (e.g., a mammal, preferably a human being) comprising administering to said patient a therapeutically effective amount of a solvate of a compound of formula 15.

Another embodiment of the present invention is directed to a method of treating psoriasis in a patient in need of such treatment (e.g., a mammal, preferably a human being) comprising administering to said patient a therapeutically effective amount of a solvate of a compound of formula 15, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 15, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 16.

Another embodiment of the present invention is directed to a method of treating psoriasis in a patient in need of such treatment (e.g., a mammal, preferably a human being) comprising administering to said patient a therapeutically effective amount of a solvate of a compound of formula 16.

Another embodiment of the present invention is directed to a method of treating psoriasis in a patient in need of such treatment (e.g., a mammal, preferably a human being) comprising administering to said patient a therapeutically effective amount of a solvate of a compound of formula 16, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 16, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 17.

Another embodiment of the present invention is directed to a method of treating psoriasis in a patient in need of such treatment (e.g., a mammal, preferably a human being) comprising administering to said patient a therapeutically effective amount of a solvate of a compound of formula 17.

Another embodiment of the present invention is directed to a method of treating psoriasis in a patient in need of such treatment (e.g., a mammal, preferably a human being) comprising administering to said patient a therapeutically effective amount of a solvate of a compound of formula 17, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 17, wherein said solvate is a monohydrate.

Another embodiment of the present invention is directed to a method of treating psoriasis in a patient in need of such treatment (e.g., a mammal, preferably a human being) comprising administering to said patient a therapeutically effective amount of a compound of formula 18.

Another embodiment of the present invention is directed to a method of treating psoriasis in a patient in need of such treatment (e.g., a mammal, preferably a human being) comprising administering to said patient a therapeutically effective amount of a solvate of a compound of formula 18.

Another embodiment of the present invention is directed to a method of treating psoriasis in a patient in need of such treatment (e.g., a mammal, preferably a human being) comprising administering to said patient a therapeutically effective amount of a solvate of a compound of formula 18, wherein said solvate is a hydrate.

Another embodiment of the present invention is directed to a method of treating psoriasis in a patient in need of such treatment (e.g., a mammal, preferably a human being) comprising administering to said patient a therapeutically effective amount of a solvate of a compound of formula 18, wherein said solvate is a monohydrate.

The compounds of this invention are useful for treating asthma. Asthma, as those skilled in the art will appreciate, includes conditions known as mild asthma, moderate asthma and severe asthma. Those skilled in the art will also appreciate that different eosinophil concentrations and/or neutrophil concentrations may be related to different asthma conditions. Therefore, the methods of treating asthma of this invention also include the treatment of asthma categorized by the associated eosinophil and/or neutrophil concentrations. Thus, the methods of treating asthma of this invention are directed to the treatment of any of the conditions of asthma, and therefore the methods of this invention include, for example, methods of treating mild asthma, methods of treating moderate asthma, and methods of treating severe asthma (including methods of treating neutrophilic asthma).

Another embodiment of the present invention is directed to a method of treating asthma in a patient in need of such treatment (e.g., a mammal, preferably a human being) comprising administering to said patient a therapeutically effective amount of at least one (e.g., 1-3, and usually one) compound selected from the group consisting of compounds of formulas 1 to 18 (or pharmaceutically acceptable salts, esters or solvates thereof).

Another embodiment of the present invention is directed to a method of treating asthma in a patient in need of such treatment (e.g., a mammal, preferably a human being) comprising administering to said patient a therapeutically effective amount of a compound selected from the group consisting of compounds of formulas 1 to 18. Another embodiment of the present invention is directed to a method of treating asthma in a patient in need of such treatment (e.g., a mammal, preferably a human being) comprising administering to said patient a therapeutically effective amount of a solvate of a compound selected from the group consisting of compounds of formulas 1 to 18.

Another embodiment of the present invention is directed to a method of treating asthma in a patient in need of such treatment (e.g., a mammal, preferably a human being) comprising administering to said patient a therapeutically effective amount of a solvate of a compound selected from the group consisting of compounds of formulas 1 to 18, wherein said solvate is a hydrate (e.g., a monohydrate).

Another embodiment of the present invention is directed to a method of treating asthma in a patient in need of such treatment (e.g., a mammal, preferably a human being) comprising administering to said patient a therapeutically effective amount of a compound of formula 1.

Another embodiment of the present invention is directed to a method of treating asthma in a patient in need of such treatment (e.g., a mammal, preferably a human being) comprising administering to said patient a therapeutically effective amount of a solvate of a compound of formula 1.

Another embodiment of the present invention is directed to a method of treating asthma in a patient in need of such treatment (e.g., a mammal, preferably a human being) comprising administering to said patient a therapeutically effective amount of a solvate of a compound of formula 1, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 1, wherein said solvate is a monohydrate.

Another embodiment of the present invention is directed to a method of treating asthma in a patient in need of such treatment (e.g., a mammal, preferably a human being) comprising administering to said patient a therapeutically effective amount of a compound of formula 2.

Another embodiment of the present invention is directed to a method of treating asthma in a patient in need of such treatment (e.g., a mammal, preferably a human being) comprising administering to said patient a therapeutically effective amount of a solvate of a compound of formula 2.

Another embodiment of the present invention is directed to a method of treating asthma in a patient in need of such treatment (e.g., a mammal, preferably a human being) comprising administering to said patient a therapeutically effective amount of a solvate of a compound of formula 2, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 2, wherein said solvate is a monohydrate.

Another embodiment at the present invention is directed to a method of treating asthma in a patient in need of such treatment (e.g., a mammal, preferably a human being) comprising administering to said patient a therapeutically effective amount of a compound of formula 3.

Another embodiment of the present invention is directed to a method of treating asthma in a patient in need of such treatment (e.g., a mammal, preferably a human being) comprising administering to said patient a therapeutically effective amount of a solvate of a compound of formula 3.

Another embodiment of the present invention is directed to a method of treating asthma in a patient in need of such treatment (e.g., a mammal, preferably a human being) comprising administering to said patient a therapeutically effective amount of a solvate of a compound of formula 3, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 3, wherein said solvate is a monohydrate.

Another embodiment of the present invention is directed to a method of treating asthma in a patient in need of such treatment (e.g., a mammal, preferably a human being) comprising administering to said patient a therapeutically effective amount of a compound of formula 4.

Another embodiment of the present invention is directed to a method of treating asthma in a patient in need of such treatment (e.g., a mammal, preferably a human being) comprising administering to said patient a therapeutically effective amount of a solvate of a compound of formula 4.

Another embodiment of the present invention is directed to a method of treating asthma in a patient in need of such treatment (e.g., a mammal, preferably a human being) comprising administering to said patient a therapeutically effective amount of a solvate of a compound of formula 4, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 4, wherein said solvate is a monohydrate.

Another embodiment of the present invention is directed to a method of treating asthma in a patient in need of such treatment (e.g., a mammal, preferably a human being) comprising administering to said patient a therapeutically effective amount of a compound of formula 5.

Another embodiment of the present invention is directed to a method of treating asthma in a patient in need of such treatment (e.g., a mammal, preferably a human being) comprising administering to said patient a therapeutically effective amount of a solvate of a compound of formula 5.

Another embodiment of the present invention is directed to a method of treating asthma in a patient in need of such treatment (e.g., a mammal, preferably a human being) comprising administering to said patient a therapeutically effective amount of a solvate of a compound of formula 5, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 5, wherein said solvate is a monohydrate.

Another embodiment of the present invention is directed to a method of treating asthma in a patient in need of such treatment (e.g., a mammal, preferably a human being) comprising administering to said patient a therapeutically effective amount of a compound of formula 6.

Another embodiment of the present invention is directed to a method of treating asthma in a patient in need of such treatment (e.g., a mammal, preferably a human being) comprising administering to said patient a therapeutically effective amount of a solvate of a compound of formula 6.

Another embodiment of the present invention is directed to a method of treating asthma in a patient in need of such treatment (e.g., a mammal, preferably a human being) comprising administering to said patient a therapeutically effective amount of a solvate of a compound of formula 6, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 6, wherein said solvate is a monohydrate.

Another embodiment of the present invention is directed to a method of treating asthma in a patient in need of such treatment (e.g., a mammal, preferably a human being) comprising administering to said patient a therapeutically effective amount of a compound of formula 7.

Another embodiment of the present invention is directed to a method of treating asthma in a patient in need of such treatment (e.g., a mammal, preferably a human being) comprising administering to said patient a therapeutically effective amount of a solvate of a compound of formula 7.

Another embodiment of the present invention is directed to a method of treating asthma in a patient in need of such treatment (e.g., a mammal, preferably a human being) comprising administering to said patient a therapeutically effective amount of a solvate of a compound of formula 7, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 7, wherein said solvate is a monohydrate.

Another embodiment of the present invention is directed to a method of treating asthma in a patient in need of such treatment (e.g., a mammal, preferably a human being) comprising administering to said patient a therapeutically effective amount of a compound of formula 8.

Another embodiment of the present invention is directed to a method of treating asthma in a patient in need of such treatment (e.g., a mammal, preferably a human being) comprising administering to said patient a therapeutically effective amount of a solvate of a compound of formula 8.

Another embodiment of the present invention is directed to a method of treating asthma in a patient in need of such treatment (e.g., a mammal, preferably a human being) comprising administering to said patient a therapeutically effective amount of a to solvate of a compound of formula 8, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 8, wherein said solvate is a monohydrate. Another embodiment of the present invention is directed to a method of treating asthma in a patient in need of such treatment (e.g., a mammal, preferably a human being) comprising administering to said patient a therapeutically effective amount of a compound of formula 9.

Another embodiment of the present invention is directed to a method of treating asthma in a patient in need of such treatment (e.g., a mammal, preferably a human being) comprising administering to said patient a therapeutically effective amount of a solvate of a compound of formula 9.

Another embodiment of the present invention is directed to a method of treating asthma in a patient in need of such treatment (e.g., a mammal, preferably a human being) comprising administering to said patient a therapeutically effective amount of a solvate of a compound of formula 9, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 9, wherein said solvate is a monohydrate.

Another embodiment of the present invention is directed to a method of treating asthma in a patient in need of such treatment (e.g., a mammal, preferably a human being) comprising administering to said patient a therapeutically effective amount of a compound of formula 10.

Another embodiment of the present invention is directed to a method of treating asthma in a patient in need of such treatment (e.g., a mammal, preferably a human being) comprising administering to said patient a therapeutically effective amount of a solvate of a compound of formula 10.

Another embodiment of the present invention is directed to a method of treating asthma in a patient in need of such treatment (e.g., a mammal, preferably a human being) comprising administering to said patient a therapeutically effective amount of a solvate of a compound of formula 10, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 10, wherein said solvate is a monohydrate.

Another embodiment of the present invention is directed to a method of treating asthma in a patient in need of such treatment (e.g., a mammal, preferably a human being) comprising administering to said patient a therapeutically effective amount of a compound of formula 11.

Another embodiment of the present invention is directed to a method of treating asthma in a patient in need of such treatment (e.g., a mammal, preferably a human being) comprising administering to said patient a therapeutically effective amount of a solvate of a compound of formula 11.

Another embodiment of the present invention is directed to a method of treating asthma in a patient in need of such treatment (e.g., a mammal, preferably a human being) comprising administering to said patient a therapeutically effective amount of a solvate of a compound of formula 11, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 11, wherein said solvate is a monohydrate.

Another embodiment of the present invention is directed to a method of treating asthma in a patient in need of such treatment (e.g., a mammal, preferably a human being) comprising administering to said patient a therapeutically effective amount of a compound of formula 12.

Another embodiment of the present invention is directed to a method of treating asthma in a patient in need of such treatment (e.g., a mammal, preferably a human being) comprising administering to said patient a therapeutically effective amount of a solvate of a compound of formula 12.

Another embodiment of the present invention is directed to a method of treating asthma in a patient in need of such treatment (e.g., a mammal, preferably a human being) comprising administering to said patient a therapeutically effective amount of a solvate of a compound of formula 12, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 12, wherein said solvate is a monohydrate.

Another embodiment of the present invention is directed to a method of treating asthma in a patient in need of such treatment (e.g., a mammal, preferably a human being) comprising administering to said patient a therapeutically effective amount of a compound of formula 13.

Another embodiment of the present invention is directed to a method of treating asthma in a patient in need of such treatment (e.g., a mammal, preferably a human being) comprising administering to said patient a therapeutically effective amount of a solvate of a compound of formula 13.

Another embodiment of the present invention is directed to a method of treating asthma in a patient in need of such treatment (e.g., a mammal, preferably a human being) comprising administering to said patient a therapeutically effective amount of a solvate of a compound of formula 13, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 13, wherein said solvate is a monohydrate.

Another embodiment of the present invention is directed to a method of treating asthma in a patient in need of such treatment (e.g., a mammal, preferably a human being) comprising administering to said patient a therapeutically effective amount of a compound of formula 14.

Another embodiment of the present invention is directed to a method of treating asthma in a patient in need of such treatment (e.g., a mammal, preferably a human being) comprising administering to said patient a therapeutically effective amount of a solvate of a compound of formula 14.

Another embodiment of the present invention is directed to a method of treating asthma in a patient in need of such treatment (e.g., a mammal, preferably a human being) comprising administering to said patient a therapeutically effective amount of a solvate of a compound of formula 14, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 14, wherein said solvate is a monohydrate.

Another embodiment of the present invention is directed to a method of treating asthma in a patient in need of such treatment (e.g., a mammal, preferably a human being) comprising administering to said patient a therapeutically effective amount of a compound of formula 15.

Another embodiment of the present invention is directed to a method of treating asthma in a patient in need of such treatment (e.g., a mammal, preferably a human being) comprising administering to said patient a therapeutically effective amount of a solvate of a compound of formula 15.

Another embodiment of the present invention is directed to a method of treating asthma in a patient in need of such treatment (e.g., a mammal, preferably a human being) comprising administering to said patient a therapeutically effective amount of a solvate of a compound of formula 15, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 15, wherein said solvate is a monohydrate.

Another embodiment of the present invention is directed to a method of treating asthma in a patient in need of such treatment (e.g., a mammal, preferably a human being) comprising administering to said patient a therapeutically effective amount of a compound of formula 16.

Another embodiment of the present invention is directed to a method of treating asthma in a patient in need of such treatment (e.g., a mammal, preferably a human being) comprising administering to said patient a therapeutically effective amount of a solvate of a compound of formula 16.

Another embodiment of the present invention is directed to a method of treating asthma in a patient in need of such treatment (e.g., a mammal, preferably a human being) comprising administering to said patient a therapeutically effective amount of a solvate of a compound of formula 16, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 16, wherein said solvate is a monohydrate.

Another embodiment of the present invention is directed to a method of treating asthma in a patient in need of such treatment (e.g., a mammal, preferably a human being) comprising administering to said patient a therapeutically effective amount of a compound of formula 17.

Another embodiment of the present invention is directed to a method of treating asthma in a patient in need of such treatment (e.g., a mammal, preferably a human being) comprising administering to said patient a therapeutically effective amount of a solvate of a compound of formula 17.

Another embodiment of the present invention is directed to a method of treating asthma in a patient in need of such treatment (e.g., a mammal, preferably a human being) comprising administering to said patient a therapeutically effective amount of a solvate of a compound of formula 17, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 17, wherein said solvate is a monohydrate.

Another embodiment of the present invention is directed to a method of treating asthma in a patient in need of such treatment (e.g., a mammal, preferably a human being) comprising administering to said patient a therapeutically effective amount of a compound of formula 18.

Another embodiment of the present invention is directed to a method of treating asthma in a patient in need of such treatment (e.g., a mammal, preferably a human being) comprising administering to said patient a therapeutically effective amount of a solvate of a compound of formula 18.

Another embodiment of the present invention is directed to a method of treating asthma in a patient in need of such treatment (e.g., a mammal, preferably a human being) comprising administering to said patient a therapeutically effective amount of a solvate of a compound of formula 18, wherein said solvate is a hydrate.

Another embodiment of the present invention is directed to a method of treating asthma in a patient in need of such treatment (e.g., a mammal, preferably a human being) comprising administering to said patient a therapeutically effective amount of a solvate of a compound of formula 18, wherein said solvate is a monohydrate.

Another embodiment of the present invention is directed to any one of the above embodiments directed to the treatment of asthma wherein the asthma treated is mild asthma.

Another embodiment of the present invention is directed to any one of the above embodiments directed to the treatment of asthma wherein the asthma treated is severe asthma (including neutrophilic asthma). Another embodiment of the present invention is directed to any one of the above embodiments directed to the treatment of asthma wherein the asthma treated is mild asthma.

Another embodiment of the present invention is directed to a method of treating acute inflammation in a patient in need of such treatment (e.g., a mammal, preferably a human being) comprising administering to said patient a therapeutically effective amount of at least one (e.g., 1-3, and usually one) compound selected from the group consisting of compounds of formulas 1 to 18 (or pharmaceutically acceptable salts, esters or solvates thereof).

Another embodiment of the present invention is directed to a method of treating chronic inflammation in a patient in need of such treatment (e.g., a mammal, preferably a human being) comprising administering to said patient a therapeutically effective amount of at least one (e.g., 1-3, and usually one) compound selected from the group consisting of compounds of formulas 1 to 18 (or pharmaceutically acceptable salts, esters or solvates thereof).

Another embodiment of the present invention is directed to a method of treating rheumatoid arthritis in a patient in need of such treatment (e.g., a mammal, preferably a human being) comprising administering to said patient a therapeutically effective amount of at least one (e.g., 1-3, and usually one) compound selected from the group consisting of compounds of formulas 1 to 18 (or pharmaceutically acceptable salts, esters or solvates thereof).

In another embodiment, the present invention provides a method of treating a chemokine mediated disease in a patient in need of such treatment (e.g., a human) comprising administering to said patient an effective amount of one or more (e.g., 1-3, and usually one) compounds selected from the group consisting of compounds of formulas 1 to 18 (or pharmaceutically acceptable salts, esters or solvates thereof) in combination with an effective amount of one or more (e.g., 1, 2 or 3, or 1 or 2, or 1) disease modifying antirheumatic drugs (DMARDs) such as, for example, methotrexate, azathioptrine, luflunomide, penicillamine, gold salts, mycophenolate, mofetil, cyclophosphamide and the like.

In another embodiment, the present invention provides a method of treating a chemokine mediated disease in a patient in need of such treatment (e.g., a human) comprising administering to said patient an effective amount of one or more (e.g., 1-3, and usually one) compounds selected from the group consisting of compounds of formulas 1 to 18 (or pharmaceutically acceptable salts, esters or solvates thereof) in combination with an effective amount of one or more nonsteroidal anti-inflammatory drugs (NSAIDs) such as, for example, piroxicam, ketoprofen, naproxen, indomethacin, ibuprofen and the like.

In another embodiment the invention provides a method of treating a chemokine mediated disease in a patient in need of such treatment (e.g., a human) comprising administering to said patient an effective amount of one or more (e.g., 1-3, and usually one) compounds selected from the group consisting of compounds of formulas 1 to 18 (or pharmaceutically acceptable salts, esters or solvates thereof) in combination with an effective amount of one or more compounds selected from the group consisting of:

(a) a disease modifying antirheumatic drug (such as, for example, methotrexate, azathioptrine, luflunomide, penicillamine, gold salts, mycophenolate, mofetil, cyclophosphamide and the like);

(b) a nonsteroidal antiinflammatory drug (such as, for example, piroxicam, ketoprofen, naproxen, indomethacin, ibuprofen and the like);

(c) COX-2 selective inhibitors such as, for example, rofecoxib and celecoxib;

(d) COX-1 inhibitors such as, for example, piroxicam;

(e) immunosuppressives such as, for example, methotrexate, cyclosporin, leflunimide, tacrolimus, rapamycin or sulfasalazine; and

(f) steroids such as, for example, betamethasone, cortisone, prednisone or dexamethasone.

In another embodiment the invention provides a method of treating a chemokine mediated disease in a patient in need of such treatment (e.g., a human) comprising administering to said patient an effective amount of one or more (e.g., 1-3, and usually one) compounds selected from the group consisting of compounds of formulas 1 to 18 (or pharmaceutically acceptable salts, esters or solvates thereof) in combination with an effective amount of one or more compounds selected from the group consisting of:

(a) a disease modifying antirheumatic drug (such as, for example, methotrexate, azathioptrine, luflunomide, penicillamine, gold salts, mycophenolate, mofetil, cyclophosphamide and the like);

(b) a nonsteroidal antiinflammatory drug (such as, for example, piroxicam, ketoprofen, naproxen, indomethacin, ibuprofen and the like);

(c) COX-2 selective inhibitors such as, for example, rofecoxib and celecoxib;

(d) COX-1 inhibitors such as, for example, piroxicam;

(e) immunosuppressives such as, for example, methotrexate, cyclosporin, leflunimide, tacrolimus, rapamycin or sulfasalazine;

(f) steroids such as, for example, betamethasone, cortisone, prednisone or dexamethasone;

(g) a biological response modifier and

(h) other anti-inflammatory agents or therapeutics useful for the treatment of chemokine mediated diseases.

In another embodiment, the invention provides a method of treating a chemokine mediated disease in a patient in need of such treatment (e.g., a human) comprising administering to said patient an effective amount of one or more (e.g., 1-3, and usually one) compounds selected from the group consisting of compounds of formulas 1 to 18 (or pharmaceutically acceptable salts, esters or solvates thereof) in combination with an effective amount of one or more biological response modifiers (BRMS) such as, for example, anti-TNF antagonists including antibodies and/or receptors/receptor fragments, IL-1 antagonists, anti-CD40, anti-CD28, IL-10, anti-adhesion molecules and the like.

In another embodiment, the invention provides a method of treating a chemokine mediated disease in a patient in need of such treatment (e.g., a human) comprising administering to said patient an effective amount of one or more (e.g., 1-3, and usually one) compounds selected from the group consisting of compounds of formulas 1 to 18 (or pharmaceutically acceptable salts, esters or solvates thereof) in combination with an effective amount of one or more compounds selected from the group consisting of:

a) anti-inflammatory agents such as, for example, p38 kinase inhibitors, PDE4 inhibitors, and TACE inhibitors;

b) chemokine receptor antagonists such as, for example, thalidomide;

c) leukotriene inhibitors; and

d) other small molecule inhibitors of pro-inflammatory cytokine production.

In another embodiment, the invention provides a method of treating a chemokine mediated disease, said disease being a pulmonary disease (e.g., COPD, asthma, or cystic fibrosis) comprising administering to a patient (e.g., a human) in need of such treatment, an effective amount of one or more (e.g., one) compounds selected from the group consisting of compounds of formulas 1 to 18 (or pharmaceutically acceptable salts, esters or solvates thereof) in combination with an effective amount of one or more compounds selected from the group consisting of: glucocorticoids, 5-lipoxygenase inhibitors, β-2 adrenoceptor agonists, muscarinic M1 antagonists, muscarinic M3 antagonists, muscarinic M2 agonists, NK3 antagonists, LTB4 antagonists, cysteinyl leukotriene antagonists, bronchodilators, PDE4 inhibitors, PDE inhibitors, elastase inhibitors, MMP inhibitors, phospholipase A2 inhibitors, phospholipase D inhibitors, histamine H₁ antagonists, histamine H3 antagonists, dopamine agonists, adenosine A2 agonists, NK1 and NK2 antagonists, GABA-b agonists, nociceptin agonists, expectorants, mucolytic agents, decongestants, antioxidants, anti-IL-8 anti-bodies, anti-IL-5 antibodies, anti-IgE antibodies, anti-TNF antibodies, IL-10, adhesion molecule inhibitors, and growth hormones. Agents that belong to these classes include, but are not limited to, beclomethasone, mometasone, ciclesonide, budesonide, fluticasone, albuterol, salmeterol, formoterol, loratadine, desloratadine, tiotropium bromide, MSI-ipratropium bromide, montelukast, theophilline, cilomilast, roflumilast, cromolyn, ZD-4407, talnetant, LTB-019, revatropate, pumafentrine, CP-955, AR-C-89855, BAY-18-8004, GW-328267, QAB-149, DNK-333, YM-40461 and TH-9506 (or pharmaceutically acceptable formulations thereof).

In another embodiment, the invention provides a method of treating a chemokine mediated disease, said disease being multiple sclerosis comprising administering to a patient in need of such treatment a therapeutically effective amount of one or more (e.g., one) compounds selected from the group consisting of compounds of formulas 1 to 18 (or pharmaceutically acceptable salts, esters or solvates thereof) in combination with an effective amount of one or more compounds selected from the group consisting of methotrexate, cyclosporin, leflunimide, sulfasalazine, β-methasone, β-interferon, glatiramer acetate, prednisone, etonercept, infliximab, and formulations thereof.

In another embodiment, the invention provides a method of treating a chemokine mediated disease, said disease being rheumatoid arthritis comprising administering to a patient in need of such treatment an effective amount of one or more (e.g., one) compounds selected from the group consisting of compounds of formulas 1 to 18 (or pharmaceutically acceptable salts, esters or solvates thereof) in combination with an effective amount of one or more compounds selected from the group consisting of a COX-2 inhibitor, a COX inhibitor, an immunosuppressive, a steroid, a PDE IV inhibitor, an anti-TNF-α compound, MMP inhibitors, glucocorticoids, chemokine inhibitors, CB2-selective inhibitors, other classes of compounds indicated for the treatment of rheumatoid arthritis, and formulations thereof.

In another embodiment, the invention provides a method of treating a chemokine mediated disease, said disease being rheumatoid arthritis comprising administering to a patient in need of such treatment an effective amount of one or more (e.g., one) compounds selected from the group consisting of compounds of formulas 1 to 18 (or pharmaceutically acceptable salts, esters or solvates thereof) in combination with an effective amount of one or more compounds selected from the group consisting of a COX-2 inhibitor, a COX inhibitor, an immunosuppressive, a steroid, a PDE IV inhibitor, an anti-TNF-α compound, MMP inhibitors, glucocorticoids, chemokine inhibitors, and CB2-selective inhibitors.

In another embodiment, the invention provides a method of treating a chemokine mediated disease, said disease being stroke and cardiac reperfusion injury comprising administering to a patient in need of such treatment an effective amount of one or more (e.g., one) compounds selected from the group consisting of compounds of formulas 1 to 18 (or pharmaceutically acceptable salts, esters or solvates thereof) in combination with an effective amount of one or more compounds selected from the group consisting of thrombolitics, antiplatelet agents, gpllb/llla antagonist, anticoagulants, other compounds indicated for the treatment of rheumatoid arthritis and formulations thereof.

In another embodiment, the invention provides a method of treating a chemokine mediated disease, said disease being stroke and cardiac reperfusion injury comprising administering to a patient in need of such treatment an effective amount of one or more (e.g., one) compounds selected from the group consisting of compounds of formulas 1 to 18 (or pharmaceutically acceptable salts, esters or solvates thereof) in combination with an effective amount of one or more compounds selected from the group consisting of thrombolitics, antiplatelet agents, gpllb/llla antagonist, and anticoagulants.

In another embodiment, the invention provides a method of treating a chemokine mediated disease, said disease being stroke and cardiac reperfusion injury comprising administering to a patient in need of such treatment an effective amount of one or more (e.g., one) compounds selected from the group consisting of compounds of formulas 1 to 18 (or pharmaceutically acceptable salts, esters or solvates thereof) in combination with an effective amount of one or more compounds selected from the group consisting of an effective amount of one or more compounds selected from the group consisting of tenecteplase, TPA, alteplase, abciximab, eftiifbatide, heparin and formulations thereof.

Examples of anti-adhesion molecules include anti-CD11a (efalizumab), CD58-Fc (alefacept), anti-VLA (natalizumab), as well as small molecule antagonists of LFA-1 (such as IC-747), VLA-4 (such as GW559090), and LFA-3. Examples of leukotriene inhibitors include LTD4 receptor antagonists (e.g., Singulair), Zileuton, and inhibitors of 5-lipoxygenase. Examples of inhibitors of cytokine production include inhibitors of TNF-α such as thalidomide. Examples of other classes of compounds indicated for the treatment of rheumatoid arthritis include inhibitors of p38 kinase, TNF-α; converting enzyme (TACE), nitric oxide synthase and methotrexate.

Another embodiment is directed to the sodium salt of a compound selected from the group consisting of the compounds of formulas 1 to 18.

Another embodiment is directed to anyone of the method of treating embodiments, or the pharmaceutical composition embodiments, wherein the compound is a sodium salt.

Another embodiment is directed to the calcium salt of a compound selected from the group consisting of the compounds of formulas 1 to 18.

Another embodiment is directed to anyone of the method of treating embodiments, or the pharmaceutical composition embodiments, wherein the compound is a calcium salt.

Certain compounds of the invention may exist in different stereoisomeric forms (e.g., enantiomers, diastereoisomers and atropisomers). The invention contemplates all such stereoisomers both in pure form and in admixture, including racemic mixtures, Isomers can be prepared using conventional methods.

Certain compounds will be acidic in nature, e.g. those compounds which possess a carboxyl or phenolic hydroxyl group. These compounds may form pharmaceutically acceptable salts. Examples of such salts may include sodium, potassium, calcium, aluminum, gold and silver salts. Also contemplated are salts formed with pharmaceutically acceptable amines such as ammonia, alkyl amines, hydroxyalkylamines, N-methylglucamine and the like.

Certain basic compounds also form pharmaceutically acceptable salts, e.g., acid addition salts. For example, the pyrido-nitrogen atoms may form salts with strong acid, while compounds having basic substituents such as amino groups also form salts with weaker acids. Examples of suitable acids for salt formation are hydrochloric, sulfuric, phosphoric, acetic, citric, oxalic, malonic, salicylic, malic, fumaric, succinic, ascorbic, maleic, methanesulfonic and other mineral and carboxylic acids well known to those skilled in the art. The salts are prepared by contacting the free base form with a sufficient amount of the desired acid to produce a salt in the conventional manner. The free base forms may be regenerated by treating the salt with a suitable dilute aqueous base solution such as dilute aqueous NaOH, potassium carbonate, ammonia and sodium bicarbonate. The free base forms differ from their respective salt forms somewhat in certain physical properties, such as solubility in polar solvents, but the acid and base salts are otherwise equivalent to their respective free base to forms for purposes of the invention.

All such acid and base salts are intended to be pharmaceutically acceptable salts within the scope of the invention and all acid and base salts are considered equivalent to the free forms of the corresponding compounds for purposes of the invention.

Compounds of this invention can exist in unsolvated and solvated forms, including hydrated forms. In general, the solvated forms, with pharmaceutically acceptable solvents such as water, ethanol and the like, are equivalent to the unsolvated forms for the purposes of this invention.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal composition can take the form of creams, lotions, aerosols and/or emulsions and can be included in a transdermal patch of the matrix or reservoir type as are conventional in the art for this purpose.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 0.01 mg to about 1000 mg, preferably from about 0.01 mg to about 750 mg, more preferably from about 0.01 mg to about 500 mg, and most preferably from about 0.01 mg to about 250 mg, according to the particular application.

The actual dosage employed may be varied depending upon the requirements of the patient and the severity of the condition being treated. Determination of the proper dosage regimen for a particular situation is within the skill of the art. For convenience, the total dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 0.04 mg/day to about 4000 mg/day, in two to four divided doses.

Classes of compounds that can be used as the chemotherapeutic agent (antineoplastic agent) include: alkylating agents, antimetabolites, natural products and their derivatives, hormones and steroids (including synthetic analogs), and synthetics. Examples of compounds within these classes are given below.

Alkylating agents (including nitrogen mustards, ethylenimine derivatives, alkyl sulfonates, nitrosoureas and triazenes): Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide.

Antimetabolites (including folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

Natural products and their derivatives (including vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, paclitaxel (paclitaxel is commercially available as Taxol® and is described in more detail below in the subsection entitled “Microtubule Affecting Agents”), Mithramycin, Deoxyco-formycin, Mitomycin-C, L-Asparaginase, Interferons (especially IFN-a), Etoposide, and Teniposide.

Hormones and steroids (including synthetic analogs): 17α-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, Zoladex.

Synthetics (including inorganic complexes such as platinum coordination complexes): Cisplatin, Carboplatin, Hydroxyurea, Amsacrine, Procarbazine, Mitotane, Mitoxantrone, Levamisole, and Hexamethylmelamine.

As used herein, a microtubule affecting agent is a compound that interferes with cellular mitosis, i.e., having an anti-mitotic effect, by affecting microtubule formation and/or action. Such agents can be, for instance, microtubule stabilizing agents or agents that disrupt microtubule formation. The microtubule affecting agents are chemotherapeutic agents.

Microtubule affecting agents useful in the invention are well known to those of skill in the art and include, but are not limited to allocolchicine (NSC 406042), Halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), Taxol® derivatives (e.g., derivatives (e.g., NSC 608832), thiocolchicine (NSC 361792), trityl cysteine (NSC 83265), vinblastine sulfate (NSC 49842), vincristine sulfate (NSC 67574), epothilone A, epothilone, and discodermolide (see Service, (1996) Science, 274:2009) estramustine, nocodazole, MAP4, and the like. Examples of such agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem. 271:29807-29812.

Particularly preferred agents are compounds with paclitaxel-like activity. These include, but are not limited to paclitaxel and paclitaxel derivatives (paclitaxel-like compounds) and analogues. Paclitaxel and its derivatives are available commercially. In addition, methods of making paclitaxel and paclitaxel derivatives and analogues are well known to those of skill in the art (see, e.g., U.S. Pat. Nos. 5,569,729; 5,565,478; 5,530,020; 5,527,924; 5,508,447; 5,489,589; 5,488,116; 5,484,809; 5,478,854; 5,478,736; 5,475,120; 5,468,769; 5,461,169; 5,440,057; 5,422,364; 5,411,984; 5,405,972; and 5,296,506).

More specifically, the term “paclitaxel” as used herein refers to the drug commercially available as Taxol® (NSC number: 125973). Taxol® inhibits eukaryotic cell replication by enhancing polymerization of tubulin moieties into stabilized microtubule bundles that are unable to reorganize into the proper structures for mitosis. Of the many available chemotherapeutic drugs, paclitaxel has generated interest because of its efficacy in clinical trials against drug-refractory tumors, including ovarian and mammary gland tumors (Hawkins (1992) Oncology, 6: 17-23, Horwitz (1992) Trends Pharmacol. Sci. 13: 134-146, Rowinsky (1990) J. Natl. Canc. Inst. 82: 1247-1259).

Additional microtubule affecting agents can be assessed using one of many such assays known in the art, e.g., a semiautomated assay which measures the tubulin-polymerizing activity of paclitaxel analogs in combination with a cellular assay to measure the potential of these compounds to block cells in mitosis (see Lopes (1997) Cancer Chemother. Pharmacol. 41:37-47).

Generally, activity of a test compound is determined by contacting a cell with that compound and determining whether or not the cell cycle is disrupted, in particular, through the inhibition of a mitotic event. Such inhibition may be mediated by disruption of the mitotic apparatus, e.g., disruption of normal spindle formation. Cells in which mitosis is interrupted may be characterized by altered morphology (e.g., microtubule compaction, increased chromosome number, etc.).

Compounds with possible tubulin polymerization activity can be screened in vitro. In a preferred embodiment, the compounds are screened against cultured WR21 cells (derived from line 69-2 wap-ras mice) for inhibition of proliferation and/or for altered cellular morphology, in particular for microtubule compaction. In vivo screening of positive-testing compounds can then be performed using nude mice bearing the WR21 tumor cells. Detailed protocols for this screening method are described by Porter (1995) Lab. Anim. Sci., 45(2):145-150.

Other methods of screening compounds for desired activity are well known to those of skill in the art. Typically such assays involve assays for inhibition of microtubule assembly and/or disassembly. Assays for microtubule assembly are described, for example, by Gaskin et al. (1974) J. Molec. Biol. 89: 737-758. U.S. Pat. No. 5,569,720 also provides in vitro and in vivo assays for compounds with paclitaxel-like activity.

Methods for the safe and effective administration of most of the chemotherapeutic agents are known to those skilled in the art. In addition, their administration is described in the standard literature. For example, the administration of many of the chemotherapeutic agents is described in the “Physicians' Desk Reference” (PDR), e.g., 2006 edition (Thompson PDR at Montvale, N.J. 07645-1742); the disclosure of which is incorporated herein by reference thereto.

The amount and frequency of administration of the compounds of this invention and the chemotherapeutic agents and/or radiation therapy will be regulated according to the judgment of the attending clinician (physician) considering such factors as age, condition and size of the patient as well as severity of the disease being treated. A dosage regimen of the compound of formula IA can be oral administration of from 10 mg to 2000 mg/day, preferably 10 to 1000 mg/day, more preferably 50 to 600 mg/day, in two to four (preferably two) divided doses, to block tumor growth. Intermittent therapy (e.g., one week out of three weeks or three out of four weeks) may also be used.

The chemotherapeutic agent and/or radiation therapy can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., antineoplastic agent or radiation) on the patient, and in view of the observed responses of the disease to the administered therapeutic agents.

In the methods of this invention, a compound of this invention is administered concurrently or sequentially with a chemotherapeutic agent and/or radiation. Thus, it is not necessary that, for example, the chemotherapeutic agent and the compound of this invention, or the radiation and the compound of this invention, should be administered simultaneously or essentially simultaneously. The advantage of a simultaneous or essentially simultaneous administration is well within the determination of the skilled clinician.

Also, in general, the compound of this invention and the chemotherapeutic agent do not have to be administered in the same pharmaceutical composition, and may, because of different physical and chemical characteristics, have to be administered by different routes. For example, the compound of this invention may be administered orally to generate and maintain good blood levels thereof, while the chemotherapeutic agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of a compound of this invention, and chemo-therapeutic agent and/or radiation will depend upon the diagnosis of the attending physicians and their judgement of the condition of the patient and the appropriate treatment protocol.

The compound of this invention, and chemotherapeutic agent and/or radiation may be administered concurrently (e.g., simultaneously, essentially simultaneously or within the same treatment protocol) or sequentially, depending upon the nature of the proliferative disease, the condition of the patient, and the actual choice of chemotherapeutic agent and/or radiation to be administered in conjunction (i.e., within a single treatment protocol) with the compound of this invention.

If the compound of this invention, and the chemotherapeutic agent and/or radiation are not administered simultaneously or essentially simultaneously, then the initial order of administration of the compound of this invention, and the chemotherapeutic agent and/or radiation, may not be important. Thus, the compound of formula IA may be administered first, followed by the administration of the chemotherapeutic agent and/or radiation; or the chemotherapeutic agent and/or radiation may be administered first, followed by the administration of the compound of this invention. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient.

For example, the chemotherapeutic agent and/or radiation may be administered first, especially if it is a cytotoxic agent, and then the treatment continued with the administration of the compound of this invention followed, where determined advantageous, by the administration of the chemotherapeutic agent and/or radiation, and so on until the treatment protocol is complete.

Thus, in accordance with experience and knowledge, the practicing physician can modify each protocol for the administration of a component (therapeutic agent—i.e., of this invention, chemotherapeutic agent or radiation) of the treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radio-logical studies, e.g., CAT or MRI scan, and successive measurements can be used to judge whether or not growth of the tumor has been retarded or even reversed, Relief of disease-related symptoms such as pain, and improvement in overall condition can also be used to help judge effectiveness of treatment.

Assay

[³⁵S]GTPγS Binding Assay:

This assay measures the inhibition of agonist (GROα) stimulated guanosine 5′-[γ-³⁵S]triphospate ([³⁵S]GTPγS, triethylammonium salt) exchange in membranes expressing CXCR2.

For each assay point, 2 μg of membrane (Baf3/hCXCR22 [Hi70]), 200 μg wheat germ agglutinin-coated SPA beads (WGA-SPA; Amersham, Arlington Heights, Ill.), 3 μM guanosine 5′-diphosphate (GDP) ±3 nM compound are pre-incubated for 3 hrs at room temperature (mixing by inversion) in SPA binding buffer (50 mM TRIS-HCL, 1 mM CaCl2, 5 mM MgCl2, 50 mM NaCl, 0.002% NaN3, 0.1% BSA, 10 μg/ml saponin, pH=7.6).

The bead, membrane, compound mixture is transferred to a 96-well Isoplate (Wallac, Gaithersburg, Md.) and pre-incubated for 60 min with concentrations of chemokine (hGROα) ranging from 500 nM to 1 nM with a no chemokine sample serving as a control. The GTPγS exchange reaction was initiated by the addition of 0.1 nM [³⁵S]GTPγS and the incubation for 60 min at room temperature. Assay is terminated by the addition of 10 μl “Stop Solution” (0.5M Na-EDTA; 10 μM GDP).

Membrane-bound [³⁵S]GTPγS was measured using a 1450 Microbeta Trilux counter (Wallac, Gaithersburg, Md.). Data is calculated as percent inhibition at the highest chemokine concentration (500 nM). The control (No compound addition), which is set to 0%, versus the % inhibition of the standard, which is set at 100%. The standard used is:

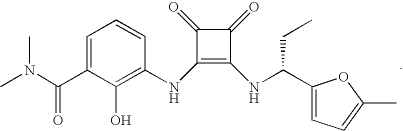

The results are given in the table below.

| | % Inhi- |
|---|---|
| | bition |
| Structure | GTPyS |
| 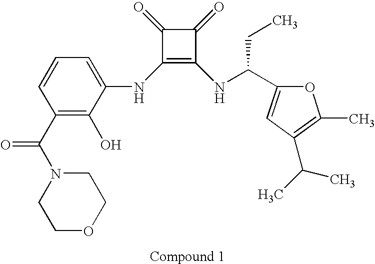 | 137 |
| 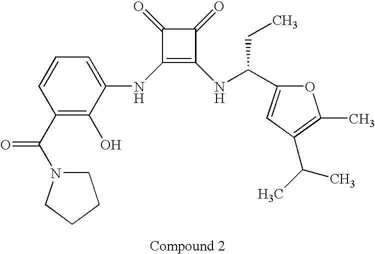 | 132 |
| 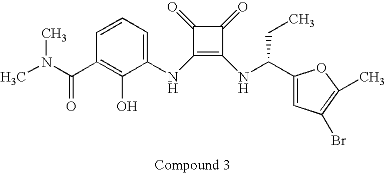 | 122 |
| 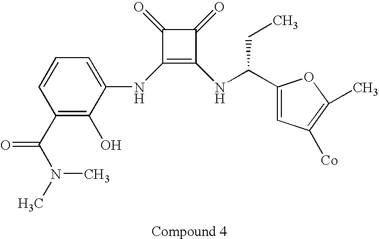 | 120 |
| 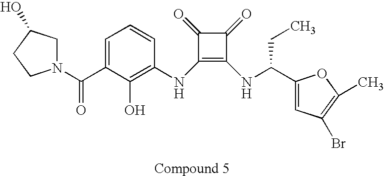 | 119 |
| 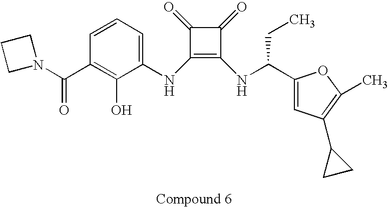 | 115 |
| 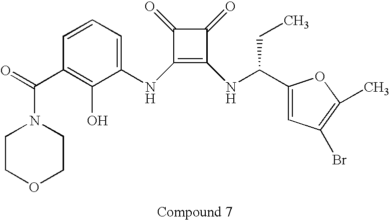 | 114 |
| 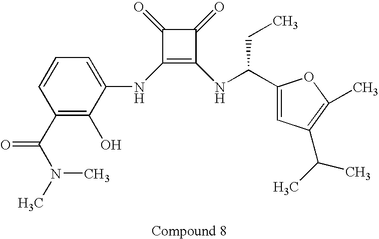 | 112 |
| 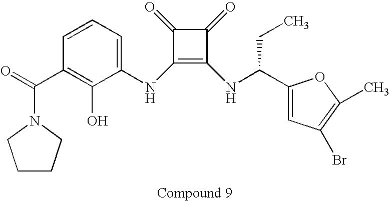 | 110 |
| 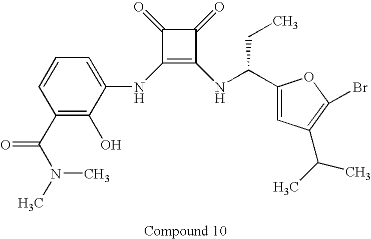 | 109 |
| 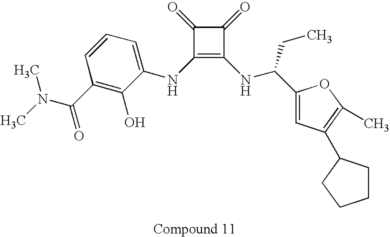 | 109 |
| 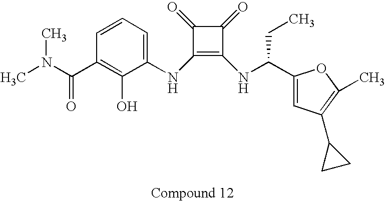 | 107 |
| 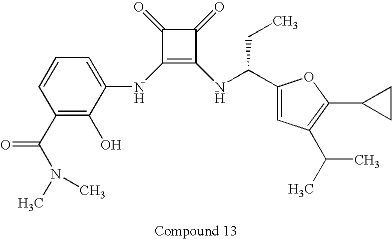 | 105 |
| 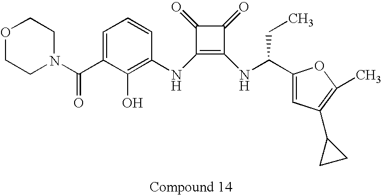 | 103 |
| 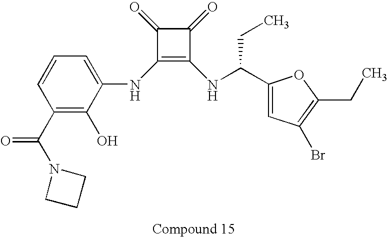 | 103 |
| 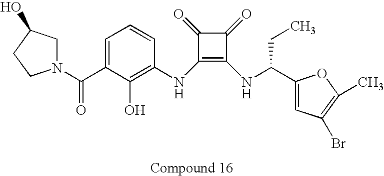 | 101 |
| 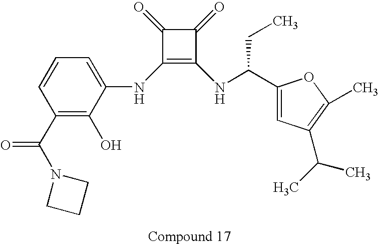 | 100 |
| 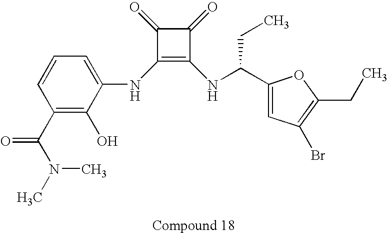 | 99 |

Compounds of this invention may be produced by using methods well known to those skilled in the art, by using the methods described and the examples below, and by using the methods disclosed in WO 02/083624 published Oct. 24, 2003 (the disclosure of which is incorporated herein by reference thereto), and using the methods disclosed in WO 2004/011418 published Feb. 5, 2004 (the disclosure of which is incorporated herein by reference thereto).

The invention disclosed herein is exemplified by the following preparations and examples, which should not be construed to limit the scope of the disclosure. Alternative mechanistic pathways and analogous structures will be apparent to those skilled in the art.

### EXAMPLE 1

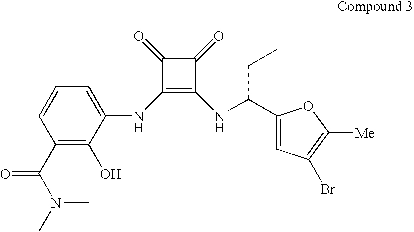 Step 1

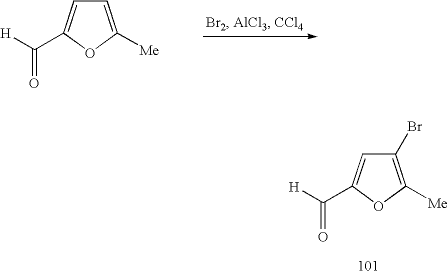

To a suspension of aluminum chloride (14.5 g, 0.11 mol) in carbon tetrachloride (60 mL) at 0° C. was added 5-methylfurfural (10 mL, 0.1 mol) with stirring under argon, followed by slow addition of a solution of bromine (5.5 mL, 0.11 mol) in carbon tetrachloride (10 mL). After addition, the resulting dark red-brown mixture was stirred at room temperature for two days when TLC analysis (EtOAc-hexanes, 3:7) showed that the reaction was complete. The mixture was then poured into ice-water (100 mL) and extracted with EtOAc (2×100 mL). The combined organic layers were washed with brine, dried over Na₂SO₄, and concentrated under reduced pressure. The residue was purified by column chromatography (EtOAc-hexanes, 5:95) to give the aldehyde 101 as a pale yellow solid (16.35 g, 86.5%) ¹H NMR (CDCl₃) δ 9.49 (s, 1H), 7.18 (s, 1H), 2.41 (s, 3H).
Step 2

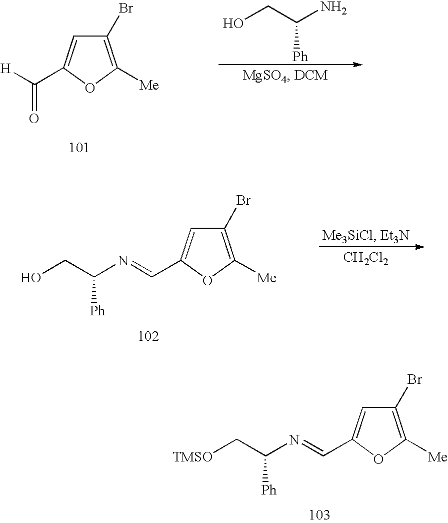

A mixture of the aldehyde 101 (820 mg, 4.34 mmol), (R)-(−)-2-phenylglycinol no (595 mg, 4.34 mmol), magnesium sulfate (1.57 g, 13.04 mmol), and dichloromethane (10 ml) was stirred at room temperature for 3 h. The mixture was filtered, and the filtrate was concentrated under reduced pressure to give the mine 102, which was used in next step without purification.

To a mixture of the imine 102 (ca. 4.34 mmol), triethylamine (1.8 mL, 13.02 mmol), and dichloromethane (20 mL) at 0° C. was added chlorotrimethylsilane (0.823 mL, 6.51 mmol) dropwise with stirring. After addition, the mixture was stirred at room temperature overnight and then concentrated under reduced pressure. The residue was mixed with ether-hexanes (1:1) (40 mL). After stirring at room temperature for 1 h, the mixture was filtered, and the filtrate was concentrated under reduced pressure to give the protected imine 103 as a brown liquid (1.5 g, 92% yield over two steps). ¹H NMR (CDCl₃) δ 8.02 (s, 1H), 7.47-7.27 (m, 5H), 6.77 (s, 1H), 4.38 (m, 1H), 3.90 (m, 2H), 2.38 (s, 3H), 0.05 (s, 9H).
Step 3

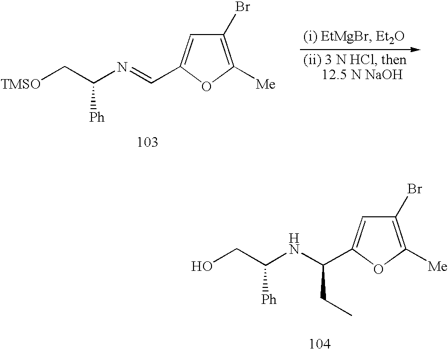

To a solution of ethylmagnesium bromide in ether (3 M, 2.7 mL, 8.1 mmol) at −42° C. was added a solution of the protected imine 103 (1.5 g, 3.99 mmol) in anhydrous ether (10 mL) dropwise with stirring under argon. After addition, the mixture was stirred for 2 h from −42° C. to room temperature. Mini-workup and ¹H NMR analysis showed that the reaction was complete. The reaction mixture was then poured slowly to a cold, saturated ammonium chloride solution (100 mL) with stirring at 0° C. Ether (150 mL) was added, and the organic layer was separated, washed with brine, dried over MgSO₄, and concentrated under reduced pressure. To the resultant residue was added ether (6 mL). After cooling to 0° C., 3 N HCl solution (20 mL) was added dropwise with stirring. The resulting mixture was stirred at room temperature for 1 h. The aqueous layer was separated and then made alkaline (pH 14) at 0° C. with 12.5 N NaOH solution. The mixture was extracted with ether (2×150 mL). The combined organic layers were dried over Na₂SO₄ and concentrated under reduced pressure to give 1.3 g (97%) of the aminoalcohol 104 as a pale brown liquid. ¹H NMR (CDCl₃) δ 7.31-7.18 (m, 5H), 6.03 (s, 1H), 3.81 (m, 1H), 3.77 (m, 1H), 3.61 (m, 2H), 2.10 (s, 3H), 1-95-1.78 (m, 2H), 0.86 (t, 3H).
Step 4

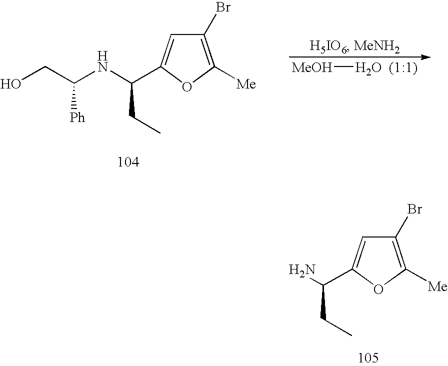

To a mixture of the aminoalcohol 104 (260 mg, 0.774 mmol), methanol (3 mL), and 40% MeNH₂ solution in water (2.1 mL) at 0° C. was added a solution of sodium periodate (1.17 g, 5.14 mmol) in water (3 mL) dropwise with stirring. The mixture was stirred at room temperature overnight. The mixture was then diluted with water (20 mL) and extracted with ether (2×50 mL). The combined organic layers were concentrated to 20 mL. 1 N HCl solution (30 mL) was added at 0° C. The resulting mixture was stirred at 0° C. for 2 h. The aqueous layer was separated and washed with ether (15 mL). The aqueous layer was made alkaline (pH˜14) at 0° C. with 12.5 N NaOH solution and extracted with ether (3×50 mL). The combined organic layers were dried over Na₂SO₄ and concentrated under reduced pressure to give 166 mg (99%) of the amine 105 as a yellow liquid. ¹H NMR (CDCl₃) δ 6.05 (s, 1H), 3.75 (m, 1H), 2.11 (s, 3H), 1.80-1.61 (m, 2H), 0.95 (t, 3H).
Step 5

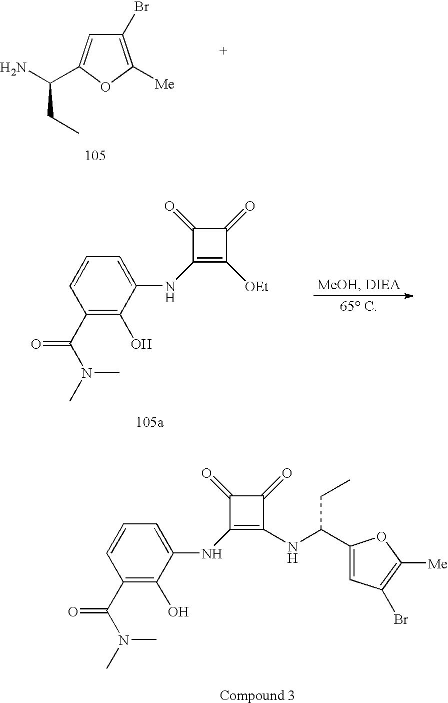

A mixture of the amine 105 (43.6 mg, 092 mmol), the intermediate 105a (see Example 2, Step 10) (60.9 mg, 0.2 mmol), diisopropylethylamine (0.2 mL), and methanol (2 mL) was stirred at 65° C. overnight when TLC analysis (CH₂Cl₂-MeOH, 9:1) showed that the starting materials disappeared. The mixture was concentrated under reduced pressure. The residue was purified by column chromatography (CH₂Cl₂-MeOH, 99:1 to 95:5) to give 70 mg (90%) of the target Compound 3 as a yellow solid. LC-MS: Rt 6.57 min, m/e 473.8, 475.8, 952.5; ¹H NMR (CDCl₃) δ 8.51 (s, 1H), 7.79 (d, 1H), 6.95 (d, 1H), 6.80 (t, 1H), 6.22 (s, 1H), 5.20 (m, 1H), 3.11 (s, 6H), 2.21 (s, 3H), 1.96-1.86 (m, 2H), 0.95 (t, 3H).

### EXAMPLE 2

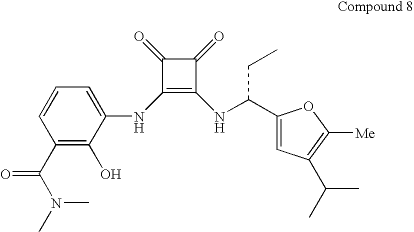 Step 1

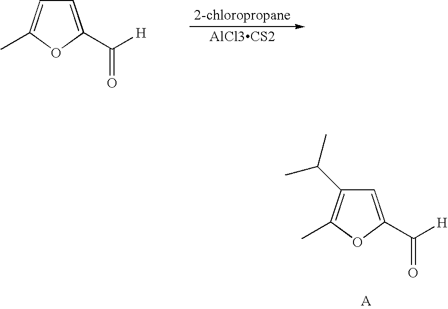

5-Methyl-furan-2-carbaldehyde (1) (2.0 moles) in CS₂ (300 ml) was added dropwise to the suspension of AlCl₃ (4 moles) in CS₂ (1.5 L) at 0° C. over 30 min. The reaction mixture was stirred at 0° C. for 15 min and at 10° C. for 1 h. The reaction mixture was carefully poured over ice-H₂O (10 L) and the aqueous layer was extracted with ether (3×4 L). The organic layer was washed with saturated NaHCO₃ (1.5 L), and H₂O (2.5 L). Dried over MgSO₄, filtered and concentrated under reduced pressure to yield a crude oil (275 g), which was purified by flash column chromatography with 0%-15% Ethyl acetate-Hexanes to provide compound (II) as a light-yellow oil 205 g (67%).
Steps 2 to 6: Intermediates B to F 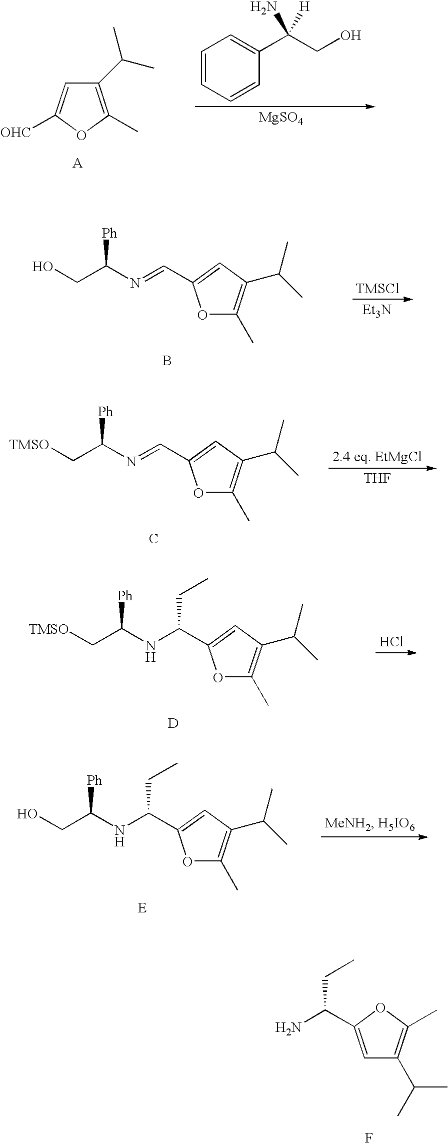
Step 2: Preparation of Compound B

MgSO₄ (6009) was added to a solution of Compound A (204 g, 1.314 mol) in dichloromethane (4 L) at room temperature. A solution of R-(−)-2-phenylglycinol (189.3 g, 1.38 mol) in dichloromethane (1.2 L) was added over 30 min. After 4 hours, MgSO₄ (200 g) was added. The mixture was stirred at room temperature over night. Solids were filtered and washed with dichloromethane (1 L). The filtrate was used directly in next reaction. ¹HNMR (CDCl₃): 8.04 (s, 1H), 7.41-7.26 (m, 5H), 6.67 (s, 1H), 4.39 (m, 1H), 4.03 (m, 1H), 3.88 (m, 1H), 2.77 (m, 1H), 2.31 (s, 3H), 1.14 (d, 6H).

Step 3: Preparation of Compound C

Triethylamine (159.6 g, 1.58 mol) and dichloromethane (157.1 g, 1.45 mol) were added to above filtrate sequentially. The mixture was stirred at room temperature for 1 hour. Hexane (4 L) was added. Solids were filtered and washed with hexane. A reddish oil (464 g) was obtained upon concentration of the filtrate. ¹HNMR (CDCl₃): 8.02 (s, 1H), 7.45-7.24 (m, 5H) 6.64 (s, 1H), 4.31 (t, 1H), 3.90 (d, 2H), 2.76 (m, 1H), 2.30 (s, 3H), 1.14 (d, 6H).

Steps 4 and 5: Preparation of Compound D and E

A solution of Compound C (454 g, 1.285 mol) in THF (1 L) was added slowly to a solution of 2M EtMgCl (1.56 L) in THF (2 L) at −35° C. It was stirred for 1 hour at −35° C. and then over night at room temperature to give Compound D.

HCl (4N, 1.8 L) was added slowly to the above mixture at 0° C. and stirred at room temperature for 3 hours. The reaction was diluted with diethylether (2 L) and hexane (3 L). The mixture was adjusted with NaOH (2N, ˜1 L) to pH ˜9. Organic layer was separated and washed with brine. Aqueous layer was acidified to pH 6 with HCl and extracted with EtOAc. All organic layers were combined and washed with brine.

A sticky oil (Compound E, 401 g) was obtained upon concentration. ¹HNMR (CDCl₃): 7.28-7.17 (m, 5H), 6.86 (s, 1H), 3.82 (m, 1H), 3.67 (m, 1H), 3.53 (m, 2H), 2.61 (m, 1H), 2.05 (s, 3H), 1.78 (m, 2H), 1.05 (d, 6H), 0.86 (t, 3H).

Step 6: Preparation of Compound F

To a solution of Compound E (401 g) in MeOH (5.3 L), was added methylamine (40% water solution, 2.2 L), followed by a solution of periodic acid (898.4 g in 1.3 L water) between 25° C. to 35° C. It was stirred over night at room temperature. Solids were filtered and washed with MeOH (0.3 L) and diethylether (0.5 L). Diethylether (4 L), water (2 L) and brine (0.3 L) were added to the filtrate. More solid was precipitated out. Solids were filtered again and washed with MeOH and ether. More diethylether (2 L) and water (1 L) were added to the filtrate. Two layers were separated. Aqueous layer was extracted with diethylether (3 L). The combined diethylether layer was washed with brine.

HCl (3N, 1 L) was added to the above diethylether layer. It was stirred at room temperature for 30 min. Two layers were separated. Diethylether layer was washed with water (0.5 L). The combined aqueous layer was basified to pH 14 with 3N NaOH and extracted with diethylether twice (2×2 L). Diethylether layer was dried with Na₂SO₄ and concentrated to an oil (262 g). Oil was loaded on a filtration plug filled with 1.1 kg of silica gel. It was eluded with 50% to 100% of ethyl acetate (EA) in hexane and finally 2% of MeOH in EA. The combined filtrate was concentrated to give Compound F as a light brown oil (191 g). ¹HNMR (CDCl₃): 5.95 (s, 1H), 3.82 (m, 1H), 3.72 (t, 1H), 2.68 (m, 1H), 2.18 (s, 3H), 1.83-1.61 (m, 2H), 1.11 (d, 6H), 0.93 (t, 3H).
Step 7

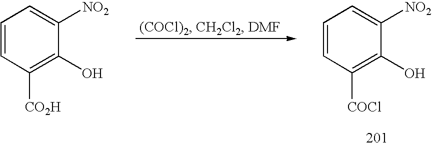

To a mixture of 3-nitrosalicylic acid (20 g, 0.109 mol), N,N-dimethylformamide (4 mL), and dichloromethane (500 mL) was added oxalyl chloride (18.6 mL, 0.225 mol, 2.06 eq) dropwise with stirring at room temperature. The reaction mixture was stirred at the same temperature for 2-3 h when all the solid in the mixture dissolved. Evaporation of solvent and excess oxalyl chloride and drying under vacuum afforded 22 g of the acid chloride 201 as a yellow solid, which was used in next step without purification.
Step 8

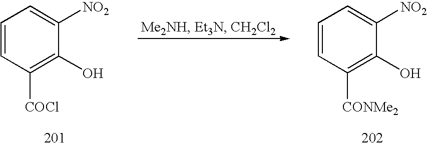

To a mixture of the acid chloride 201 (22 g, ca. 0.109 mol) and dichloromethane (400 mL) at 0° C. was added triethylamine (61 mL, 0.437 mol) slowly with stirring under argon, followed by slow addition of 2 M dimethylamine solution in tetrahydrofuran (108 mL, 0.218 mol). After addition, the mixture was stirred at room temperature overnight. The mixture was then concentrated under reduced pressure, and EtOAc (500 mL) and water (200 mL) were added. The organic layer was separated, washed with 1 N HCl solution, water, and brine, dried over Na₂SO₄, and concentrated under reduced pressure to give the amide 202 as a yellow solid (20.95 g, 91% over two steps). ¹H NMR (CDCl₃) δ 10.92 (s, 1H), 8.15 (d, 1H), 7.62 (d, 1H), 7.06 (t, 1H), 7.08 (d, 1H), 3.08 (s, 6H).
Step 9

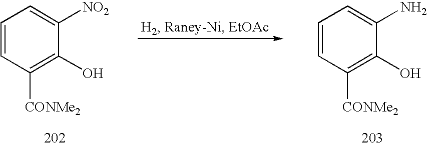

A mixture of the amide 202 (20.95 g, 99.7 mmol), EtOAc (200 mL), and Raney-nickel (3 spoons) was subjected to hydrogenation at 60 psi at room temperature overnight. The mixture was filtered through a layer of Celite. The filtrate was concentrated under reduced pressure to give the dark oil residue, which was purified by column chromatography (EtOAc-hexanes, 1:1) to give 11.13 g (62%) of the amine 203 as a colorless oil. ¹H NMR (CDCl₃) δ 6.80-6.65 (m, 3H) (d, 1H), 3.15 (s, 3H).

Step 10

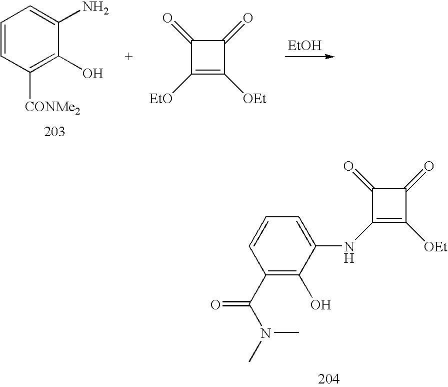

A mixture of the amine 203 (14.55 g, 80.74 mmol), EtOH (500 mL), and 3,4-diethoxy-3-cyclobutene-1,2-dione (14.4 g, 80.74 mmol) was stirred at room temperature overnight. The mixture was then concentrated under reduced pressure. The residue was purified by column chromatography (EtOAc-hexanes, 3:1) to give 20.46 g (84%) of the Compound 204 as a yellow solid. ¹H NMR (CDCl₃) δ 10.99 (s, 1H), 8.00-7.64 (m, 2H), 7.09 (d, 1H), 6.88 (t, 1H), 4.86 (q, 2H), 3.18 (s, 6H), 1.51 (t, 3H).
Step 11

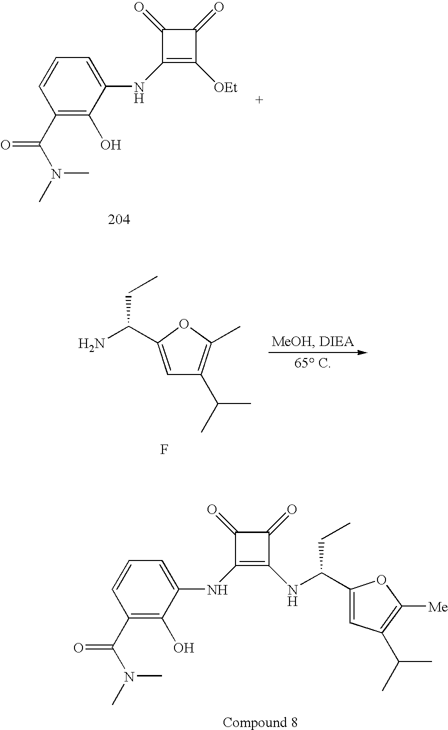

A mixture of the amine F (4.9 g, 27.07 mmol), the intermediate 204 (8 g, 26.32 mmol), diisopropylethylamine (0.6 mL), and ethanol (140 mL) was stirred at 65° C. overnight when TLC analysis (CH₂Cl₂-MeOH, 9:1) showed that the starting materials disappeared. The mixture was then concentrated under reduced pressure. The residue was purified by column chromatography (CH₂Cl₂-MeOH, 30:1) to give 8.2 g (71%) of the target Compound 8 as a pale brown solid. LC-MS: Rt 6.82 min, m/e 462.0, 900.9; ¹H NMR (DMSO-d₆) δ 9.85 (s, 1H), 9.18 (s, 1H), 8.56 (d, 1H), 7.76 (d, 1H), 6.80 (m, 2H), 6.18 (s, 1H), 5.00 (m, 1H), 3.22 (s, 1H), 2.88 (s, 6H), 2.61 (m, 1H), 2.08 (s, 3H), 1.96-1.86 (m, 2H), 1.02 (d, 6H), 0.95 (t, 3H).

Following procedures similar to those of Examples 1 and 2, Compounds 1, 2, 4-7, and 9-18 (identified above) can be prepared.

### PREPARATIVE EXAMPLE 1

### Preparation of Compound (213) (HCl salt of compound (212))

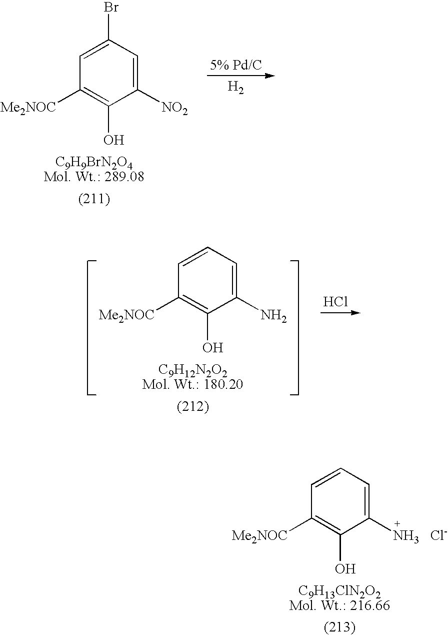

To a suspension of 10 g (34.6 mmol) of (211) in a mixture of 21 ml of methyl t-butylether and 49 ml of ethanol was added 13.7 ml of KOEt (24%) in ethanol, followed by addition of 0.8 g of 5% Pd/C (50% wet). The mixture was then agitated under 120-150 psi hydrogen pressure for about 6 hours. Upon completion of the reaction, the batch was filtered through a Celite pad and the cake was washed with 80 ml of solvent mixture of methyl t-butylether and ethanol (1:1). The filtrate was treated with 3.7 ml of concentrated HCl solution. The batch was then concentrated under reduced pressure to about 50 ml. Isopropanol (100 ml) was added and the resulting solution was concentrated under vacuum to about 40 ml. Methyl t-butylether (50 ml) was added, followed by a slow addition of 110 ml of heptane. Finally, the mixture was cooled to 0° C. The solids were collected by filtration and the cake was washed with 20 ml solvent mixture of 1:1 methyl t-butylether/EtOH. The cake was dried at 60° C. for 10 hours in a vacuum oven, to give 7.24 g (96%) off-white solids. ¹H NMR (DMSO-D6): 7.50 (d, 1H), 6.96 (dd, 1H), 7.17 (d, 1H), 2.9 (br, 6H), 10.2 (br, 4H), ¹³C NMR (DMSO-D6): 147.7, 121.4, 125.9, 120.6, 128.5, 127.1, 167.8.

### PREPARATIVE EXAMPLE 2

### Preparation of the Oxalate of Compound (212)

Following the procedure described for preparing the HCl salt (213) in Preparative Example 1, 10 g (34.6 mmol) of compound (211) was hydrogenated under the same condition and the filtered solution was treated with 3.3 g of oxalic acid. Following the same procedure as above resulted in 8.5 g (90%) off-white solids. ¹H NMR (DMSO-D6): 6.45 (m, 2H), 6.17 (dd, 1H), 2.70 (s, 6H). 5.5 (very broad, 4H).

### PREPARATIVE EXAMPLE 3

### Preparation of the p:PTSA Salt of Compound (212)

Following the procedure described for preparing the HCl salt (213) in Preparative Example 1, 10 g of compound (211) was hydrogenated under the same condition and the filtrate was treated with 7.9 g (41.1 mmol) p-toluenesulfonic acid monohydrate. The resulting mixture was concentrated as above and the mixture after heptane addition was stirred over night at room temperature, to give 11.4 g (94%) off-white solids. ¹H NMR (DMSO-D6): 7.49 (d, 2H), 7.29 (d, 1H), 7.15 (m, 3H), 6.93 (dd, 1H), 2.90 (s, 6H), 2.31 (s, 3H).

### PREPARATIVE EXAMPLE 4

### Preparation of Tartrate of Compound (212)

Following the procedure described for preparing the HCl salt (213) in Preparative Example 1, 10 g of compound (211) was hydrogenated under the same condition and the filtrate was treated with 5.47 g (36.5 mmol) of tartaric acid. Following the same procedure as described in 527123-PS preparation resulted in 9.1 g (80%) of off-white solids. ¹H NMR (DMSO-D6): 8.5 (br, 3H), 6.6 (dd, 2H), 6.38 (d, 1H), 4.26 (s, 2H), 3.6 (b, 2H), 2.96 (s, 6H).

### PREPARATIVE EXAMPLE 5

### Preparation of Compound 209A

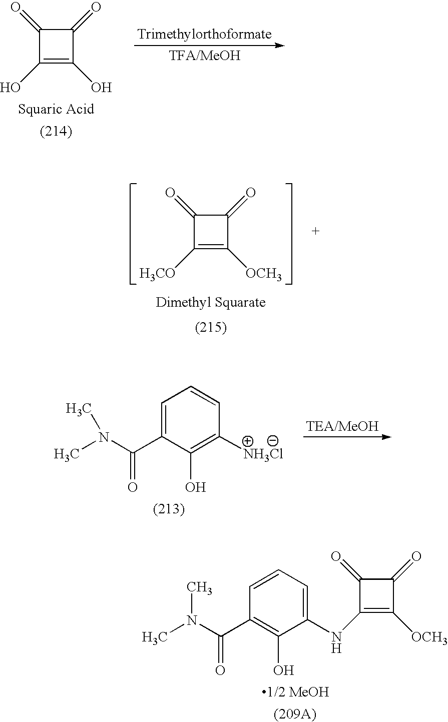

Charged 9.5 kg of the compound of formula 214 to 50 gallon glass reactor equipped with a thermocouple, N₂ inlet and feed tank, Charged 65 liters dry methanol (KF<0.1%) followed by 20 liters trimethylorthoformate and 0.2 kg trifluororacetic acid. Heated the batch to reflux and maintained for about one hour. Concentrated the batch at one atmosphere until the internal temperature exceeded 70° C. Maintained the batch at reflux for about four hours. Adjusted the batch to a temperature between 40 and 50° C. and charged 26 liters dry methanol. Adjusted the temperature to about 20 to 30° C. Charged 78 liters of dry methanol and adjusted the batch to a temperature between −5 and 5° C. Charged 13.0 kg of the compound of formula V. Over about 4 hours, charged 11.1 kg triethylamine (TEA) to the batch while maintaining the batch at a temperature between −5 and 5° C. About one and a half hours after the start of the TEA charge, seeded the batch with 130 grams of compound (209A) added as a solid. After the addition of TEA was completed, agitated the batch for about 30 minutes at a temperature between −5 and 5° C. Charged 12 liters acetic acid while maintaining the batch at a temperature between −5 and 5° C. Heated the batch to a temperature between 60 and 70° C. and maintained this temperature for about 1 hour. Adjusted the temperature to about 25 to 35° C. over about 1 hour. Adjusted the temperature to about −5 to 5° C. over about 1 hour. Filtered the batch and washed the filter cake with 65 liters (5×) methanol. Dried the batch in a vacuum oven for at least 24 hours at 60 to 70° C. Yield 14.5 kg, 81%. ¹HNMR (CD₃CN) 8.07 (1H, s); 7.56 (1H, d); 7.28 (1H, d); 6.99 (1H, t); 4.35 (3H, s); 3.10 (6H, s)

### PREPARATIVE EXAMPLE 6

### Preparation of Compound (209A) from Dimethylsquarate and Compound (213)

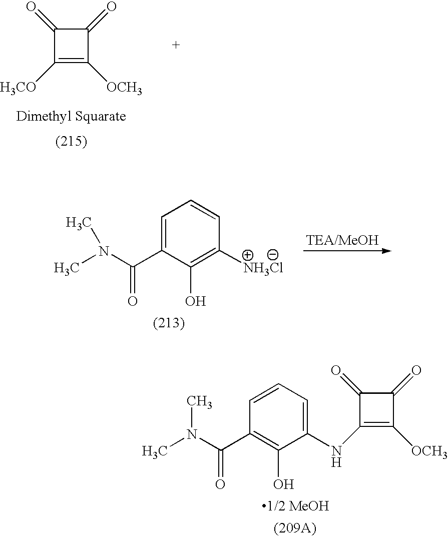

Charged 6.3 grams of compound (213) and 5.0 grams of compound (215) to 250 ml round bottom flask equipped with a thermocouple, N₂ inlet and addition funnel. Charged 41 ml dry methanol (KF<0.1%). Adjusted the batch to temperature between −5 and 5° C. Over about 5 hours, charged 4.9 ml triethylamine (TEA) to the batch while maintaining the batch at a temperature between −5 and 500. After the addition of TEA was completed, agitated the batch for about one hour at a temperature between −5 and 5° C. Charged 2.8 ml acetic acid while maintaining the batch at a temperature between −5 and 5° C. Adjusted the batch volume to 63 ml by adding dry methanol. Heated the batch to reflux and maintained for about 15 minutes. Adjusted the temperature to about −5 to 5° C. over about 1 hour. Filtered the batch and washed the filter cake with 25 ml (5×) methanol. Dried the batch in a vacuum oven for at least 24 hours at 60 to 70° C. Yield 7.5 g, 88%.

### PREPARATIVE EXAMPLE 7

### Preparation of Compound (2098) from Diethylsquarate (216) and Compound (213)

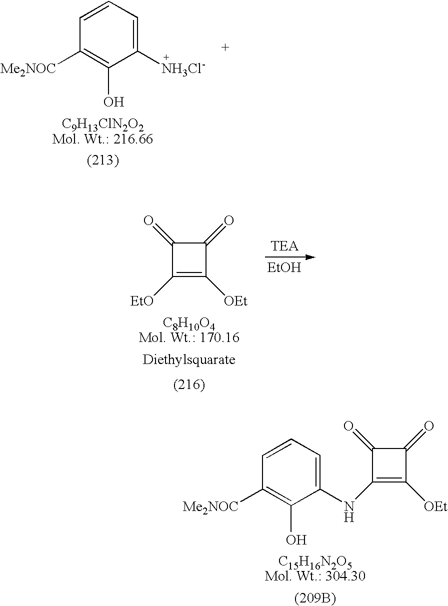

Charged 44.0 kg of the compound (213), 225 kg dry ethanol and 41.8 kg of the compound (216) to a 300 gallon glass lined reactor equipped with a thermocouple, N₂ inlet and feed bottle. Adjusted the batch to temperature between 0 and 10° C. Over about 1 hour, charged 17.1 kg triethylamine (TEA) to the batch while maintaining the batch at a temperature between 0 and 10° C. After the addition of TEA was complete, agitated the batch for about three hours at a temperature between 0 and 10° C. Over about 3 hours, charged additional 8.2 kg triethylamine (TEA) to the batch while maintaining the batch at a temperature between 0 and 10° C. After the addition of TEA was complete, agitated the batch for about three hours at a temperature between 0 and 10° C. Charged 19 liters acetic acid while maintaining the batch at a temperature between 0 and 10° C. Adjusted the batch volume to 440 liters by adding dry ethanol. Heated the batch to reflux and maintained for about 15 minutes. Adjusted the temperature to about 0 to 10° C. over about 2 hours. Filtered the batch and washed the filter cake with 220 liters 50% v/v ethanol in water. Dried the batch in a vacuum oven for at least 12 hours at 50 to 60° C. Yield 52 kg, 88%.

¹HNMR (CD₃CN) 7.61 (1H, d); 7.28 (1H, d); 6.96 (1H, t); 4.69 (2H, q); 3.10 (6H, s), 1.44 (3H, t).

### EXAMPLE 3

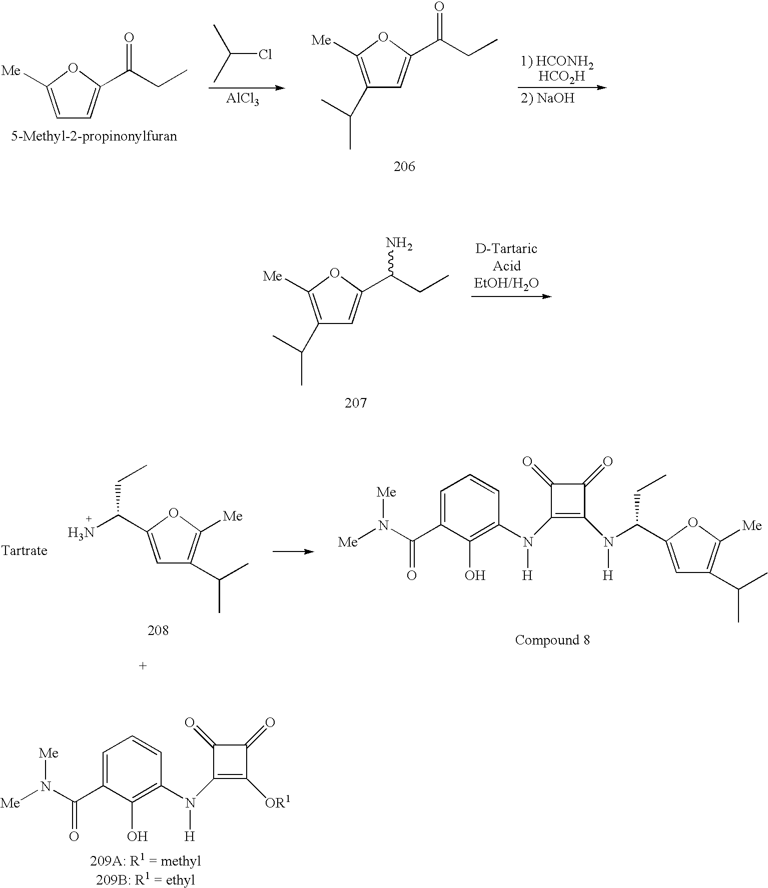

### Step 1: 1-(4-isopropyl-5-methyl-2-furyl)propan-1-one (206)

Under nitrogen, 2-methyl-5-propionylfurane (100 g, 0.72 moles) was added dropwise at 0-30° C. to aluminium chloride (131 g, 0.96 moles). The resulting suspension was stirred for further 30 minutes at room temperature and then cooled to 0-5° C. Within one hour isopropyl chloride (76 g, 0.96 moles) was added dropwise at 0-10° C. and the mixture stirred until complete conversion was achieved (HPLC). The mixture was hydrolyzed on 2 L of water/ice. The pH was adjusted to 1 by addition of sodium hydroxide solution (60 mL) and the product was extracted into 500 mL TBME. The aqueous layer was separated and reextracted with 200 mL TBME. The combined organic layers were washed with 500 mL brine and evaporated to minimum volume. Yield: 132.5 g (102%) of a yellow-brown liquid.

Assay (HPLC: YMC Pack Pro C18 150×4.6 mm, 5 μm; 220 nm; ACN/0.05% TFA: water/0.05% TFA 20:80 to 95:5 within 23 min): 60% pure by area, RT 17.2 min.

### Step 2: [1-(4-Isopropyl-5-methyl-2-furyl)propyl]amine (207)

Under nitrogen, a mixture of crude 1-(4-isopropyl-5-methyl-2-furyl)propan-1-one (100 g), formamide (100 g, 2.22 moles) and formic acid (28.7 g, 0.61 moles) was heated to 140° C. for about two days until complete conversion to intermediate N-(1-(4-isopropyl-5-methylfuran-2-ylpropyl)formamide was achieved. The mixture was cooled to 20-25° C. and diluted with 400 mL methanol and 400 mL diisopropylether Aqueous sodium hydroxide (1.2 kg, 25% in water) was added and the mixture was heated to reflux (55-60° C.) for about one day until complete conversion to [1-(4-Isopropyl-5-methyl-2-furyl)propyl]amine was achieved. The mixture was cooled down to 20-25° C. and the phases were separated. The organic layer was washed with 400 mL brine (5% in water). The combined aqueous layers were reextracted with 200 mL diisopropylether. The combined organic layers were evaporated to minimum volume. Yield: 94.6 g (45% abs (absolute) from 2-methyl-5-propionylfurane) of a yellow-brown liquid.

Assay (HPLC: YMC Pack Pro C18 150×4.6 mm, 5 μm; 220 nm; ACN/0.05% TFA: water/0.05% TFA 20:80 to 95:5 within 23 min): 48.5% pure vs. standard, RT 9.2 min.

### Step 3: (R)-1-(4-Isopropyl-5-methylfuran-2-yl)propan-1-amine (2S,3S)-2,3-dihydroxysuccinate (208)

Under nitrogen, crude [1-(4-isopropyl-5-methyl-2-furyl)propyl]amine (51 g, 135 mmol active) was dissolved in 204 mL dry ethanol at 60° C. 20% of a solution of D-(−)-tartaric acid (20.3 g, 135 mmol) in a mixture of 102 mL ethanol/water (15:1) was added at 55° C. The solution was seeded. The residual solution of tartaric acid was added within 10 minutes. The suspension was cooled to 20° C. and stirred at room temperature over night. The salt was filtered off and washed with dry ethanol until a colorless mother liquor was obtained. The product was dried in vacuum at 50° C. to constant weight. Yield: 16.9 g (38% abs.) of white crystals.

Assay (HPLC: YMC Pack Pro C18 150×4.6 mm, 5 μm; 220 nm; ACN:0.01 M KH₂PO₄ pH=2.5 (H₃PO₄) 15:85 to 80:20 within 25 min): 95.8% by area, RT 8.8 min.

Optical Purity (HPLC: Chiralcel OD-R 250×4.6 mm; 226 nm; ACN:0.5M NaClO₄ 40:60): dr 98:2, RT 12.6 min (R), 16.3 min (S). Wherein “dr” represents diastereomeric ratio.

### Step 4: 2-Hydroxy-3-[(2-{[(1R)-1-(4-isopropyl-5-methyl-2-furyl)propyl]amino}-3,4-dioxocyclobut-1-en-1-yl)amino]-N,N-dimethylbenzamide (Compound 8)

Under nitrogen, (R)-1-(4-Isopropyl-5-methylfuran-2-yl)propan-1-amine (2S,3S)-2,3-dihydroxy-succinate (208) (2.0 g, 6 mmol) was suspended in 6 ml water and 8 mL 2-methyl tetrahydrofurane (MeTHF) at 20-25° C. 1.3 mL aqueous sodium hydroxide (30%) were added and the organic layer was separated after 5 minutes. The aqueous layer was extracted with 4 mL MeTHF. The combined organic layers were added to (209B) (1.74 g, 5.7 mmol) and 4 mL MeTHF were added. The mixture was heated to 65° C. for 4.5 hours and was then cooled to 20-25° C. After 16 hours at 20-25° C. the product crystallized and was isolated by filtration. The product was washed with MeTHF and dried in vacuum at 5000 to constant weight. Yield: 1.25 g (47%) as off-white solid. Assay (NMR): 95% pure.

If one were to use compound (209A) in Example 3 then one would obtain compound (8).

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

What is claimed is:

1. A compound selected from the group consisting of compounds of the formula: 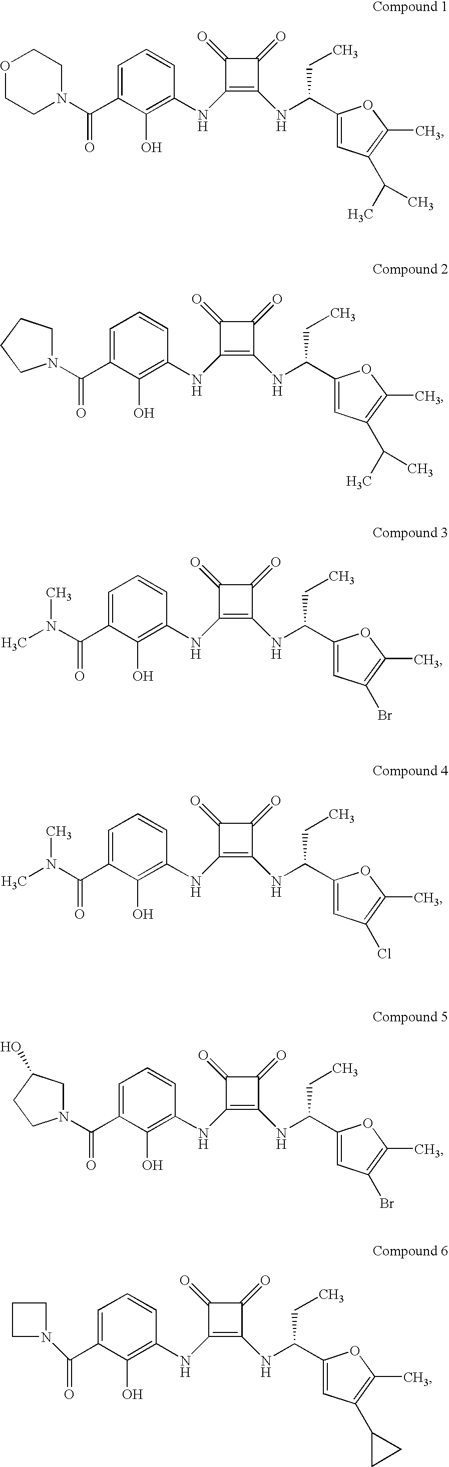 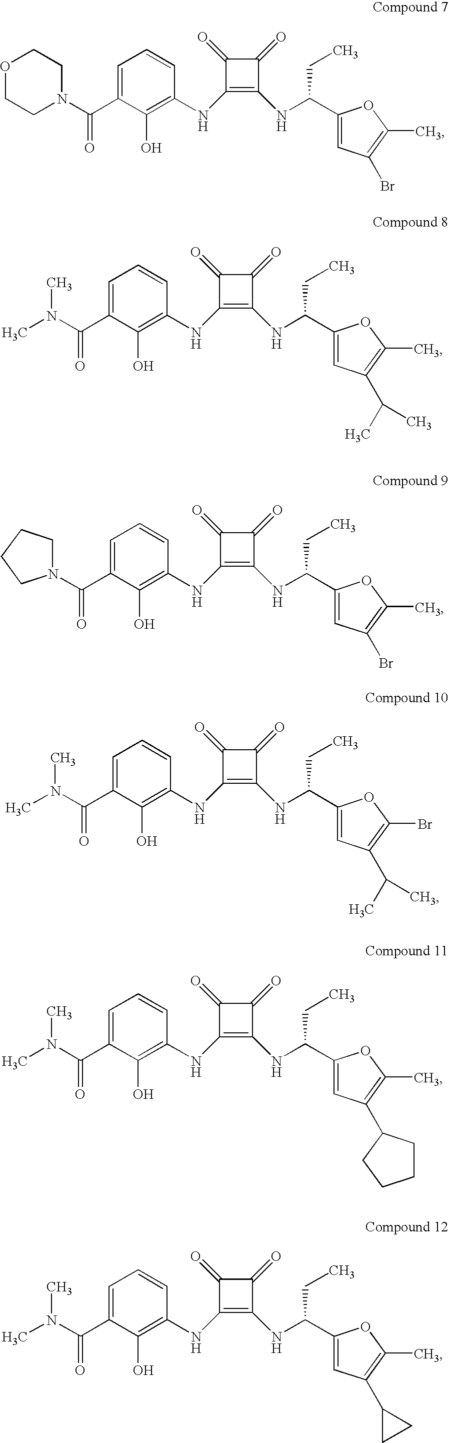 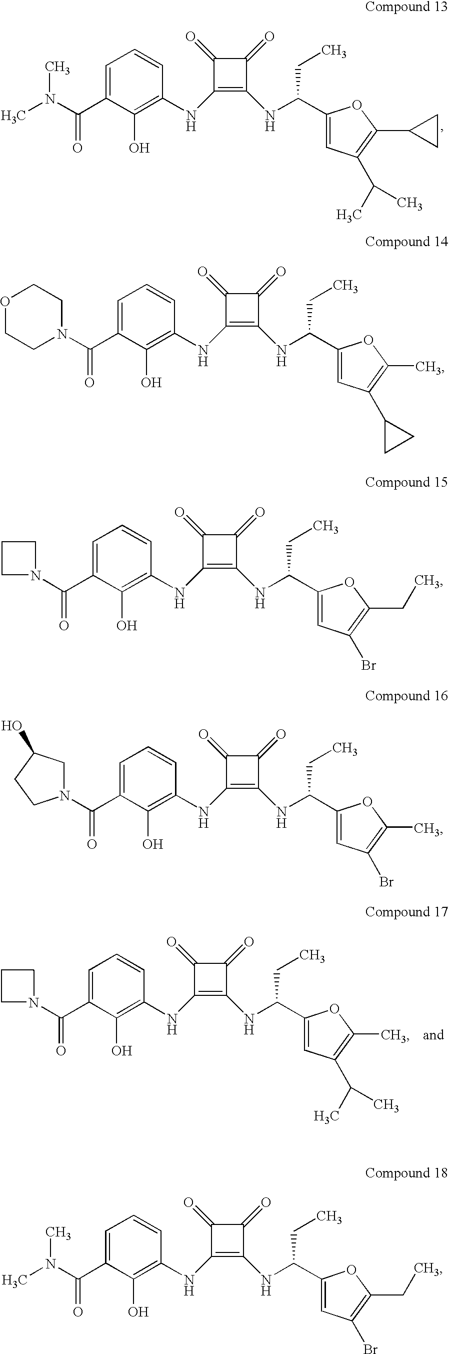
or the pharmaceutically acceptable salts, esters and solvates thereof.

2. The compound of claim 1 selected from the group consisting of compounds of the formula:

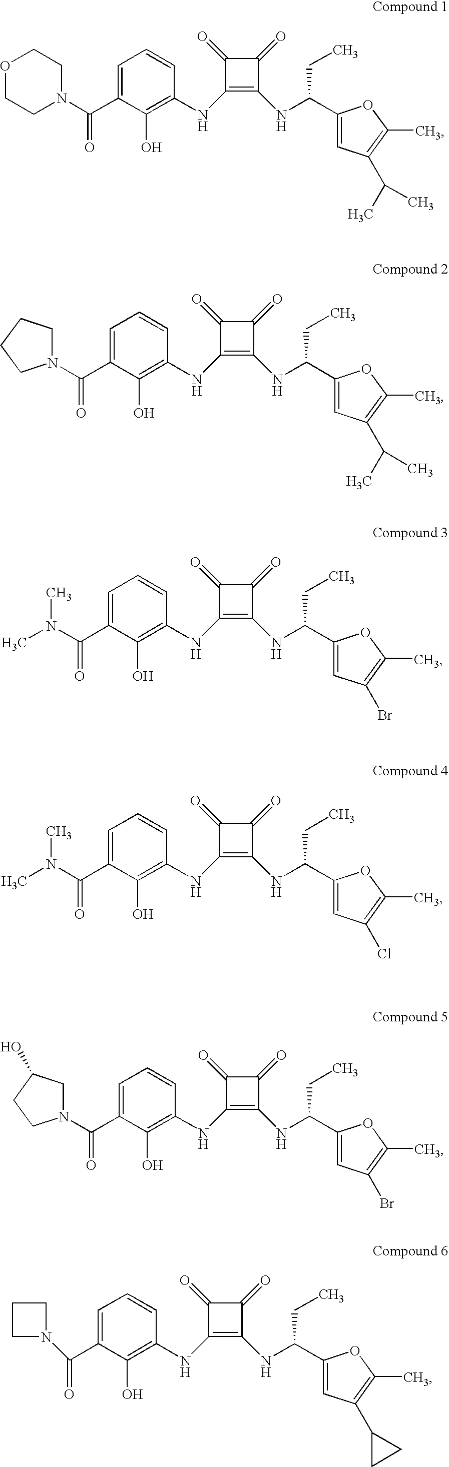

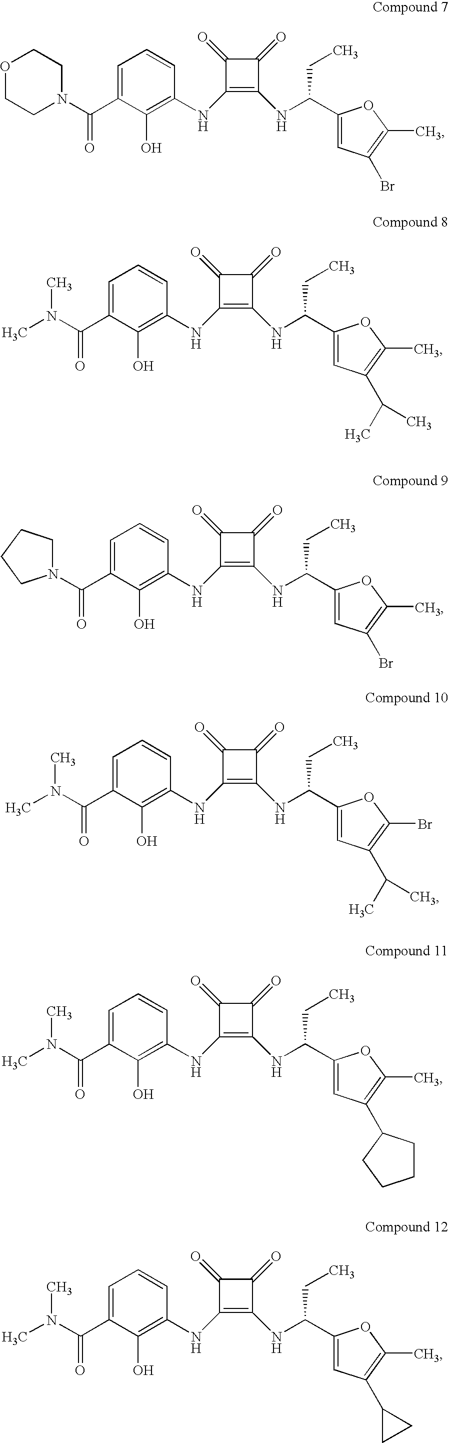

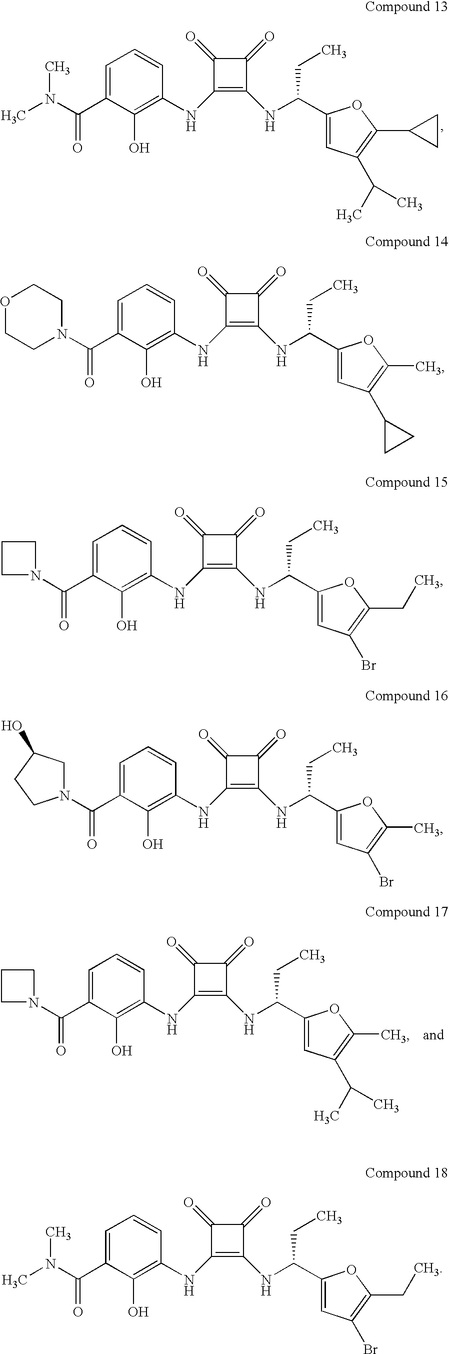

3. The compound of claim 1 wherein said compound is a pharmaceutically acceptable salt.

4. The compound of claim 1 wherein said compound is Compound 1.

5. The compound of claim 1 wherein said compound is Compound 2.

6. The compound of claim 1 wherein said compound is Compound 3.

7. The compound of claim 1 wherein said compound is Compound 4.

8. The compound of claim 1 wherein said compound is Compound 5.

9. The compound of claim 1 wherein said compound is Compound 6.

10. The compound of claim 1 wherein said compound is Compound 7.

11. The compound of claim 1 wherein said compound is Compound 8.

12. The compound of claim 1 wherein said compound is Compound 9.

13. The compound of claim 1 wherein said compound is Compound 10.

14. The compound of claim 1 wherein said compound is Compound 11.

15. The compound of claim 1 wherein said compound is Compound 12.

16. The compound of claim 1 wherein said compound is Compound 13.

17. The compound of claim 1 wherein said compound is Compound 14.

18. The compound of claim 1 wherein said compound is Compound 15.

19. The compound of claim 1 wherein said compound is Compound 16.

20. The compound of claim 1 wherein said compound is Compound 17.

21. The compound of claim 1 wherein said compound is Compound 18.

22. The compound of claim 1 wherein said compound is a solvate.

23. The compound of claim 1 wherein said compound is a solvate of Compound 1.

24. The compound of claim 1 wherein said compound is a solvate of Compound 2.

25. The compound of claim 1 wherein said compound is a solvate of Compound 3.

26. The compound of claim 1 wherein said compound is a solvate of Compound 4.

27. The compound of claim 1 wherein said compound is a solvate of Compound 5.

28. The compound of claim 1 wherein said compound is a solvate of Compound 6.

29. The compound of claim 1 wherein said compound is a solvate of Compound 7.

30. The compound of claim 1 wherein said compound is a solvate of Compound 8.

31. The compound of claim 1 wherein said compound is a solvate of Compound 9.

32. The compound of claim 1 wherein said compound is a solvate of Compound 10.

33. The compound of claim 1 wherein said compound is a solvate of Compound 11.

34. The compound of claim 1 wherein said compound is a solvate of Compound 12.

35. The compound of claim 1 wherein said compound is a solvate of Compound 13.

36. The compound of claim 1 wherein said compound is a solvate of Compound 14.

37. The compound of claim 1 wherein said compound is a solvate of Compound 15.

38. The compound of claim 1 wherein said compound is a solvate of Compound 16.

39. The compound of claim 1 wherein said compound is a solvate of Compound 17.

40. The compound of claim 1 wherein said compound is a solvate of Compound 18.

41. The compound of claim 1 wherein said compound is a solvate and said solvate is a hydrate.

42. The compound of claim 1 wherein said compound is a monohydrate of Compound 1.

43. The compound of claim 1 wherein said compound is a monohydrate of Compound 2.

44. The compound of claim 1 wherein said compound is a monohydrate of Compound 3.

45. The compound of claim 1 wherein said compound is a monohydrate of Compound 4.

46. The compound of claim 1 wherein said compound is a monohydrate of Compound 5.

47. The compound of claim 1 wherein said compound is a monohydrate of Compound 6.

48. The compound of claim 1 wherein said compound is a monohydrate of Compound 7.

49. The compound of claim 1 wherein said compound is a monohydrate of Compound 8.

50. The compound of claim 1 wherein said compound is a monohydrate of Compound 9.

51. The compound of claim 1 wherein said compound is a monohydrate of Compound 10.

52. The compound of claim 1 wherein said compound is a monohydrate of Compound 11.

53. The compound of claim 1 wherein said compound is a monohydrate of Compound 12.

54. The compound of claim 1 wherein said compound is a monohydrate of Compound 13.

55. The compound of claim 1 wherein said compound is a monohydrate of Compound 14.

56. The compound of claim 1 wherein said compound is a monohydrate of Compound 15.

57. The compound of claim 1 wherein said compound is a monohydrate of Compound 16.

58. The compound of claim 1 wherein said compound is a monohydrate of Compound 17.

59. The compound of claim 1 wherein said compound is a monohydrate of Compound 18.

60. A pharmaceutical composition comprising at least one compound of claim 1 and a pharmaceutically acceptable carrier.

61. A pharmaceutical composition comprising at least one compound of claim 2 and a pharmaceutically acceptable carrier.

62. A pharmaceutical composition comprising at least one compound of claim 22 and a pharmaceutically acceptable carrier.

63. A pharmaceutical composition comprising at least one compound of claim 41 and a pharmaceutically acceptable carrier.

64. A pharmaceutical composition comprising a compound of claim 1 and a pharmaceutically acceptable carrier.

65. A pharmaceutical composition comprising a compound of claim 2 and a pharmaceutically acceptable carrier.

66. A pharmaceutical composition comprising a compound of claim 22 and a pharmaceutically acceptable carrier.

67. A pharmaceutical composition comprising a compound of claim 41 and a pharmaceutically acceptable carrier.

68. A method of treating, or inhibiting, a chemokine mediated disease in a patient in need of such treatment comprising administering to said patient an effective amount of at least one compound of claim 1, or a pharmaceutically acceptable salt, ester or solvate thereof.

69. A method of treating, or inhibiting, a chemokine mediated disease in a patient in need of such treatment comprising administering to said patient an effective amount a compound of claim 1, or a pharmaceutically acceptable salt, ester or solvate thereof.

70. The method of claim 69 wherein a compound of claim 1 is administered.

71. The method of claim 69 wherein a salt of a compound of claim 1 is administered.

72. The method of claim 69 wherein a solvate of a compound of claim 1 is administered.

73. The method of claim 72 wherein said solvate is a hydrate.

74. The method of claim 73 wherein said hydrate is a monohydrate.

75. The method of claim 69 wherein said chemokine mediated disease is cancer.

76. The method of claim 69 wherein said chemokine mediated disease is cancer and said compound is administered concurrently or sequentially with (a) an antineoplastic agent, or (b) an anti-angiogenesis agent, or (c) a VEGF receptor kinase inhibitor, or (d) antibodies against the VEGF receptor, or (e) interferon, and/or (f) radiation.

77. The method of claim 69 wherein the chemokine mediated disease inhibited is angiogenesis.

78. The method of claim 69 wherein the chemokine mediated disease is angiogenic ocular disease.

79. The method of claim 69 wherein the chemokine mediated disease is selected from the group consisting of: gingivitis, respiratory viruses, herpes viruses, hepatitis viruses, HIV, kaposi's sarcoma associated virus and atherosclerosis.

80. The method of claim 69 wherein said chemokine mediated disease is selected from the group consisting of: acute inflammatory pain, chronic inflammatory pain, acute neuropathic pain, and chronic neuropathic pain.

81. The method of claim 68 wherein said chemokine mediated disease is COPD.

82. The method of claim 69 wherein said chemokine mediated disease is COPD.

83. The method of claim 82 wherein the compound of claim 1 is a solvate.

84. The method of claim 83 wherein said solvate is a hydrate.

85. The method of claim 84 wherein said hydrate is a monohydrate.

86. The method of claim 68 wherein said chemokine mediated disease is psoriasis.

87. The method of claim 69 wherein said chemokine mediated disease is psoriasis.

88. The method of claim 87 wherein the compound of claim 1 is a solvate.

89. The method of claim 88 wherein said solvate is a hydrate.

90. The method of claim 89 wherein said hydrate is a monohydrate.

91. The method of claim 68 wherein said chemokine mediated disease is asthma.

92. The method of claim 69 wherein said chemokine mediated disease is asthma.

93. The method of claim 92 wherein the compound of claim 1 is a solvate.

94. The method of claim 93 wherein said solvate is a hydrate.

95. The method of claim 94 wherein said hydrate is a monohydrate.

96. The method of claim 68 wherein said chemokine mediated disease is severe asthma.

97. The method of claim 69 wherein said chemokine mediated disease is severe asthma.

98. The method of claim 92 wherein the compound of claim 1 is a solvate.

99. The method of claim 93 wherein said solvate is a hydrate.

100. The method of claim 94 wherein said hydrate is a monohydrate.

101. The method of claim 69 wherein said chemokine mediated disease is acute inflammation or chronic inflammation.

102. The method of claim 69 wherein said chemokine mediated disease is rheumatoid arthritis.

103. The method of claim 69 wherein said chemokine mediated disease is selected from the group consisting of: acute inflammation, chronic inflammation, rheumatoid arthritis, acute inflammatory pain, chronic inflammatory pain, acute neuropathic pain, chronic neuropathic pain, psoriasis, atopic dermatitis, asthma, COPD, adult respiratory disease, arthritis, inflammatory bowel disease, Crohn's disease, ulcerative colitis, septic shock, endotoxic shock, gram negative sepsis, toxic shock syndrome, stroke, cardiac and renal reperfusion injury, glomerulonephritis, thrombosis, Alzheimer's disease, graft vs. host reaction, allograft rejections, malaria, acute respiratory distress syndrome, delayed type hypersensitivity reaction, atherosclerosis, cerebral and cardiac ischemia, osteoarthritis, multiple sclerosis, restinosis, angiogenesis, osteoporosis, gingivitis, respiratory viruses, herpes viruses, hepatitis viruses, HIV, Kaposi's sarcoma associated virus, meningitis, cystic fibrosis, pre-term labor, cough, pruritis, multi-organ dysfunction, trauma, strains, sprains, contusions, psoriatic arthritis, herpes, encephalitis, CNS vasculitis, traumatic brain injury, CNS tumors, subarachnoid hemorrhage, post surgical trauma, interstitial pneumonitis, hypersensitivity, crystal induced arthritis, acute and chronic pancreatitis, acute alcoholic hepatitis, necrotizing enterocolitis, chronic sinusitis, angiogenic ocular disease, ocular inflammation, retinopathy of prematurity, diabetic retinopathy, macular degeneration with the wet type preferred and corneal neovascularization, polymyositis, vasculitis, acne, gastric and duodenal ulcers, celiac disease, esophagitis, glossitis, airflow obstruction, airway hyperresponsiveness, bronchiectasis, bronchiolitis, bronchiolitis obliterans, chronic bronchitis, cor pulmonae, cough, dyspnea, emphysema, hypercapnea, hyperinflation, hypoxemia, hyperoxia-induced inflammations, hypoxia, surgical lung volume reduction, pulmonary fibrosis, pulmonary hypertension, right ventricular hypertrophy, peritonitis associated with continuous ambulatory peritoneal dialysis (CAPD), granulocytic ehrlichiosis, sarcoidosis, small airway disease, ventilation-perfusion mismatching, wheeze, colds, gout, alcoholic liver disease, lupus, burn therapy, periodontitis, transplant reperfusion injury and early transplantation rejection.

104. A method of treating, or inhibiting, a chemokine mediated disease in a patient in need of such treatment comprising administering to said patient an effective amount of:
at least one compound selected from the group consisting of: 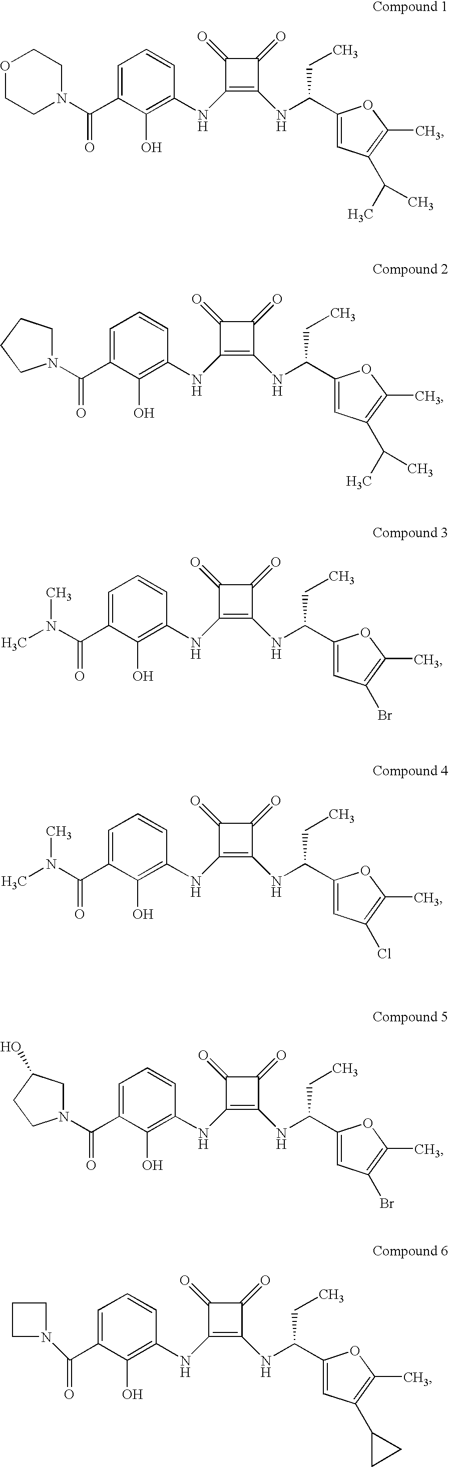 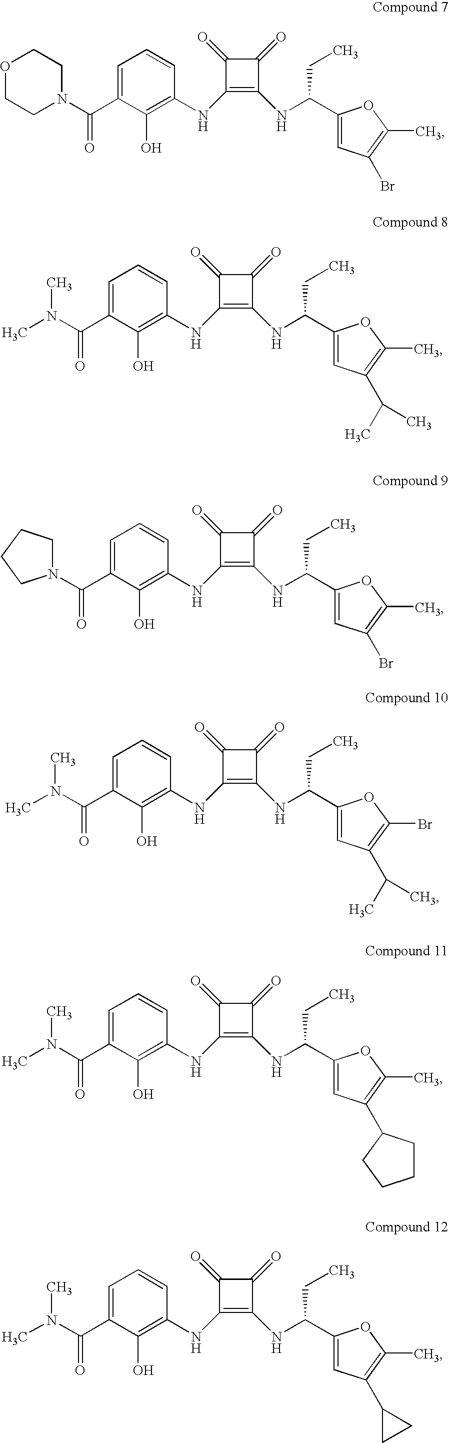 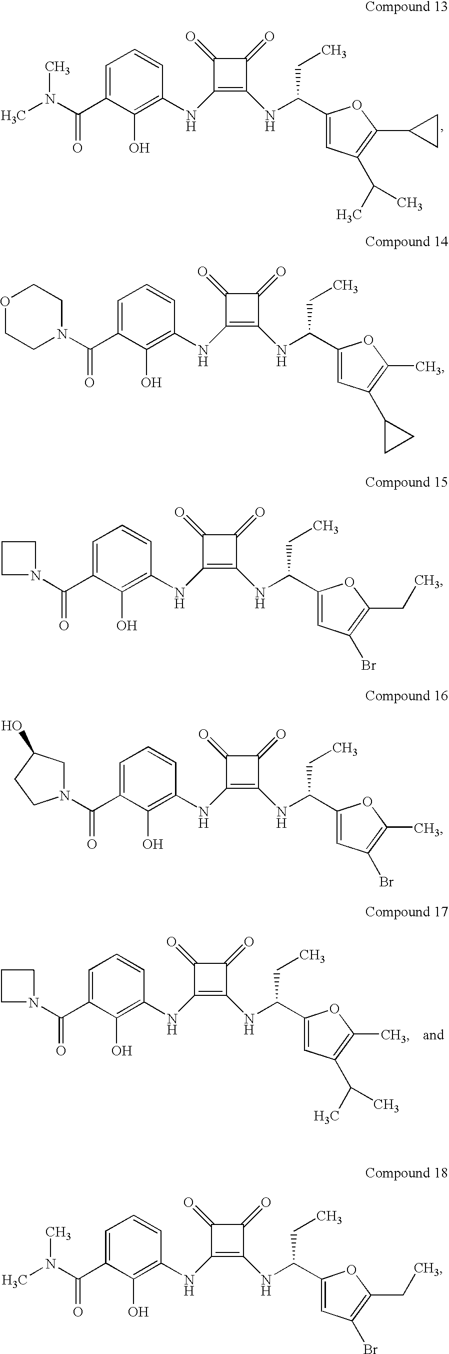
or the pharmaceutically acceptable salts, esters and solvates thereof;
in combination with:
one or more drugs, agents or therapeutics useful for the treatment of chemokine mediated diseases.

105. The method of claim 104 wherein said drug, agent or therapeutic is selected from the group consisting of: (a) a disease modifying antirheumatic drug; (b) a nonsteroidal antiinflammatory drug; (c) a COX-2 selective inhibitor; (d) a COX-1 inhibitor; (e) an immunosuppressive; (f) a steroid; (g) a biological response modifier and (h) other anti-inflammatory agents or therapeutics useful for the treatment of chemokine mediated diseases.

106. The method of claim 105 wherein said disease modifying antirheumatic drug is selected from the group consisting of methotrexate, azathioptrine luflunomide, penicillamine, gold salts, mycophenolate, mofetil and cyclophosphamide.

107. The method of claim 105 wherein said nonsteroidal antiinflammatory drug is selected from the group consisting of piroxicam, ketoprofen, naproxen, indomethacin, and ibuprofen.

108. The method of claim 105 wherein said COX-2 selective inhibitor is selected from the group consisting of rofecoxib and celecoxib.

109. The method of claim 105 wherein said COX-1 inhibitor is piroxicam.

110. The method of claim 105 wherein said immunosuppressive is selected from the group consisting of methotrexate, cyclosporin, leflunimide, tacrolimus, rapamycin and sulfasalazine.

111. The method of claim 105 wherein said steroid is selected from the group consisting of β-methasone, prednisone, cortisone, prednisolone and dexamethasone.

112. The method of claim 105 wherein said biological response modifier is selected from the group consisting of anti-TNF antagonists, IL-1 antagonists, anti-CD40, anti-CD28, IL-10 and anti-adhesion molecules.

113. The method of claim 105 wherein said other anti-inflammatory agents or therapeutics are selected from the group consisting of p38 kinase inhibitors, PDE4 inhibitors, TACE inhibitors, chemokine receptor antagonists, thalidomide, leukotriene inhibitors and other small molecule inhibitors of pro-inflammatory cytokine production.

114. The method of claim 105 wherein said chemokine mediated disease is selected from the group consisting of psoriasis, atopic dermatitis, asthma, COPD, adult respiratory disease, arthritis, inflammatory bowel disease, Crohn's disease, ulcerative colitis, septic shock, endotoxic shock, gram negative sepsis, toxic shock syndrome, stroke, cardiac and renal reperfusion injury, glomerulonephritis, thrombosis, Alzheimer's disease, graft vs. host reaction, allograft rejections, malaria, acute respiratory distress syndrome, delayed type hypersensitivity reaction, atherosclerosis, cerebral and cardiac ischemia, osteoarthritis, multiple sclerosis, restinosis, angiogenesis, osteoporosis, gingivitis, respiratory viruses, herpes viruses, hepatitis viruses, HIV, Kaposi's sarcoma associated virus, meningitis, cystic fibrosis, pre-term labor, cough, pruritis, multi-organ dysfunction, trauma, strains, sprains, contusions, psoriatic arthritis, herpes, encephalitis, CNS vasculitis, traumatic brain injury, CNS tumors, subarachnoid hemorrhage, post surgical trauma, interstitial pneumonitis, hypersensitivity, crystal induced arthritis, acute and chronic pancreatitis, acute alcoholic hepatitis, necrotizing enterocolitis, chronic sinusitis, angiogenic ocular disease, ocular inflammation, retinopathy of prematurity, diabetic retinopathy, macular degeneration with the wet type preferred and corneal neovascularization, polymyositis, vasculitis, acne, gastric and duodenal ulcers, celiac disease, esophagitis, glossitis, airflow obstruction, airway hyperresponsiveness, bronchiectasis, bronchiolitis, bronchiolitis obliterans, chronic bronchitis, cor pulmonae, cough, dyspnea, emphysema, hypercapnea, hyperinflation, hypoxemia, hyperoxia-induced inflammations, hypoxia, surgical lung volume reduction, pulmonary fibrosis, pulmonary hypertension, right ventricular hypertrophy, peritonitis associated with continuous ambulatory peritoneal dialysis (CAPD), granulocytic ehrlichiosis, sarcoidosis, small airway disease, ventilation-perfusion mismatching, wheeze, colds, gout, alcoholic liver disease, lupus, burn therapy, periodontitis and early transplantation.

115. The method of claim 105 wherein said chemokine mediated disease is a pulmonary disease and said one or more drugs, agents or therapeutics are selected from the group consisting of: glucocorticoids, 5-lipoxygenase inhibitors, β-2 adrenoceptor agonists, muscarinic M1 and M3 antagonists, muscarinic M2 agonists, NK3 antagonists, LTB4 antagonists, cysteinyl leukotriene antagonists, bronchodilators, PDE4 inhibitors, PDE inhibitors, elastase inhibitors, MMP inhibitors, phospholipase A2 inhibitors, phospholipase D inhibitors, histamine H₁ antagonists, histamine H3 antagonists, dopamine agonists, adenosine A2 agonists, NK1 and NK2 antagonists, GABA-b agonists, nociceptin agonists, expectorants, mucolytic agents, decongestants, antioxidants, anti-IL-8 anti-bodies, anti-IL-5 antibodies, anti-IgE antibodies, anti-TNF antibodies, IL-10, adhesion molecule inhibitors, and growth: hormones.

116. The method of claim 115 wherein said pulmonary disease is COPD, asthma or cystic fibrosis.

117. The method of claim 105 wherein said chemokine mediated disease is multiple sclerosis and said one or more drugs, agents or therapeutics are selected from the group consisting of methotrexate, cyclosporin, leflunimide, sulfasalazine, β-methasone, β-interferon, glatiramer acetate, prednisone, etonercept, and infliximab.

118. The method of claim 105 wherein said chemokine mediated disease is rheumatoid arthritis and said one or more drugs, agents or therapeutics are selected from the group consisting of a COX-2 inhibitor, a COX inhibitor, an immunosuppressive, a steroid, a PDE IV inhibitor, an anti-TNF-α compound, MMP inhibitors, glucocorticoids, chemokine inhibitors, CB2-selective inhibitors, and other classes of compounds indicated for the treatment of rheumatoid arthritis.

119. The method of claim 105 wherein said chemokine mediated disease is stroke and cardiac reperfusion injury and said one or more drugs, agents or therapeutics are selected from the group consisting of thrombolitics, antiplatelet agents, gpllb/llla antagonist, anticoagulants, other compounds indicated for the treatment of rheumatoid arthritis and formulations thereof.

120. The method of claim 105 wherein said chemokine mediated disease is stroke and cardiac reperfusion injury and said one or more drugs, agents or therapeutics are selected from the group consisting of tenecteplase, TPA, alteplase, abciximab, eftiifbatide, heparin and formulations thereof.
